SEQUENCE LISTING (26 sequences)

SEQ ID NO: 1 - DNA, 1458 bp, Deinococcus sp.
  CDS (1)..(1458)
atgcgccgccttcccctcctcgccgcgctgctcgcctcgctggcaggcgcgcaggcctcccccaccctcccgtccttcgaggggcaggtgatctatcaggtgatgcctgaccggttttttgacgggaacaaggcgaacgacgcaggggtcgaccgctcggacccacgtgcctggcacggcggcgatctggcgggcctcacggccaaactgccctacctccggcagctgggggcgacggcggtatggctgacgccaatctaccggcagcagacggccaacgccttcggtaccgccccctaccacggctactggcccgccgacttccgcgacgtggacccacatttcgggacactggccgattttgtcggtttcgtcaaggcggcacacggggcggggctgcgcgtggtcctcgatcaggtgatcaaccattacggctacgaggcggcggcggtcaaggaacacccggcctggttcaatgggaaagcagcgtgcgacgcttccggcaacaaggacgtgaactgcccactggcgggcctgccggacctcaagcagtcgaaccccgaggtcagggcgctgctgctgggcaacgccgacttctggcgggggcagggggtggatggtttccgctacgacgcgatcaagaacgtggagacgccctttctgaaagagctgttggcacgcgaccgtgccgccgggacgtggacgctgggcgagtggtacggggcagacaccgggacggtggccgactggcagcaggccgggttcgacagtctcttcctcttcagcctgcaacaggcgatggggcagagcctgatgggcgggcagggcctcagccgggtggcgagcgtgctgagccgccaaggcgagctgccacgccccggcgaggtggccctctttctggacaaccacgatgtcccgcgctttgcccagggcagcctgttcgaggaccaggcccaggcccgcacccgctacggcctgcgtgcgctgatgaccctgaagggcgtgccggtgctctggcagggcactgagattgccatgcgcggcggtcccgaccccgataaccgccgcgatatgcgcttcgagaacgagtggacccctgccgagcgccaggtcttcgagacggcccgggacgccatcgccgtccgccaggccagccgggccctcagcatcgggacccagaagctgctccccacacccgcctccctggaagacgacctgctcctcttcacacgcgaggcgcagggggagcgcgtgctggtcgcctggcacaacggcaggaaccgcaagacgtacagcctccgcctgagcgccctgggactgaaagcggagccgcaggccgtcacccgcagcctcttcgccgggcaggacgccaagctcagcgtgagcgggggctggctgcacctgagtctgcctgcggaggacgcggcaggatttgggctggggggaagcacccggtga

SEQ ID NO: 2 - protein, 485 aa, Deinococcus sp.
MRRLPLLAALLASLAGAQASPTLPSFEGQVIYQVMPDRFFDGNKANDAGVDRSDPRAWHGGDLAGLTAKLPYLRQLGATAVWLTPIYRQQTANAFGTAPYHGYWPADFRDVDPHFGTLADFVGFVKAAHGAGLRVVLDQVINHYGYEAAAVKEHPAWFNGKAACDASGNKDVNCPLAGLPDLKQSNPEVRALLLGNADFWRGQGVDGFRYDAIKNVETPFLKELLARDRAAGTWTLGEWYGADTGTVADWQQAGFDSLFLFSLQQAMGQSLMGGQGLSRVASVLSRQGELPRPGEVALFLDNHDVPRFAQGSLFEDQAQARTRYGLRALMTLKGVPVLWQGTEIAMRGGPDPDNRRDMRFENEWTPAERQVFETARDAIAVRQASRALSIGTQKLLPTPASLEDDLLLFTREAQGERVLVAWHNGRNRKTYSLRLSALGLKAEPQAVTRSLFAGQDAKLSVSGGWLHLSLPAEDAAGFGLGGSTR

SEQ ID NO: 3 - DNA, 3066 bp, Deinococcus sp.
  CDS (1)..(3066)
atgaaacgtttccagaaggtgggtcgcagtggcgccctggccgtccttacgttggctctgtccgcctgtggcgtcttgaaggcgcccgagacgggaggcaacactcgtgcctggcaggacgaggtgatctacttcgccatgaccgaccgcttcgccaacgggaacccggccaacgacaacggcccgaaccgcaatgagggcgaccgggccgaccggaccaacccgctcggctggcacggcggcgacttcgcggggctgaaggcgaagatcgaggagggctatttcaagcgcatgggctttacggccctctggatcagcccggtggtcctgcaggttccggccatcgagggcccgaagaccgggccgaacgccgggaagctcttcgcgggataccacggctactgggccgaggactttttcaaggtagacccacacttcggcacgctggacgagtacaagtccctcatccagactgcgcacaggaacggcatcaaggtgattcaggacattgtggtcaaccacgcgggctacggcgccacactcaccaagaccaatcctgactggtttcacacccaggctgaatgcgacgccagcaccaacaaacgggtggactgtccgctggcgggcctgcctgacttcaagcaggagcggcccgaggtcacaacgtacctgaacgacttcgtgaactcctggcgcaaggaaaccggcatcgacgggctgcggatcgacaccatgcagcacgtctctgacagctactggcagcagttctttgccgcgggtgggccgggggacccttccaagatctggtcggtcggcgaggtgttcaacggtgatccggccttcctggcccactatatggatgacctcggatcgcccagcgtgttcgatttcgcgctgtacttcgccatcaaggatggcttgtcgagtgcgcgcggcgacctaggacgcttggccgacgtgttcgcgcgggatggtgcgtaccgggaccccacacggctgaccaccttcgtggacaaccacgacgtgccccgcttcgtgagcgaggtgcaggagcgcggcgggacagcggcgcaggcgaacgagcgccttgacctggccctcagtctgatctatacctcgcgcggcacaccgagcgtgtaccagggcacggagatcgcgcagcctggcttgggcgacccctacaactacgccaccggccaaggcaaccgcgaggacatgaacttcggggccctctcgcagagcagtatcgacgagcggctggcagctctcgccgcggcacgcgcgaagtaccgggcactcacacatggcgtgcagcaggagctgtggcggccaaacggcggggcgcccatcttcgcccaccgccggattgtcacggatggtcaaggcggacagcccgtcgtcgtcgtgatcaacaacggcgacacgcccgtggacctctccactctgagcgggggcggtattccgctgctggggaccttcagcgggacggcgctgacagaaattaccgggcgaaccagcgacctgagcgtgagcggcggccaactcgtaggcacggttcctgcccgctccgcgcttgctgtcacggccccggcgggcagcggcagcacaggcacggtgaaccccaggctgccggaggtgacggatctcagtgcgaaggccggagacagcgccgtgcagctcacgtggacggcctccacggacctgaacgtcaccggctgccgcgtctacgcccgcaccgggagcgggcaggaacggctcctcaacttcgcgccgctgcccaaggaccagaccacgtacctcgccgcaggcattccgaacgaccaggaaacgaccttccgggtggtcacggtagacgcgcagggcgccgagagtcggggcgtcagcgtcaaggccacgcccagcagcaagaacacggtcagggtgactttcacggtggacgcccgcagccagggcaacggcccgatcgagctgcgccgcttcgacacgggctcgcagcttgagtaccccatgacgcaggtgagccgcggcatctggaagacggcgattgacctccccctcttccgcgagatcaagtttaagttcggcaacgacggacccgccgccaagaacagcggctacgaggcacccggccaacccgaccgcagctatgtggtgggaacaaatcctaacgtctacaccggcacctatgactttattacccagccggtgccgcagaccaccatcgagggccaagtcagaggagcgggcaatcccctcgcgaatgcgttggtcgaagcggtgaccgccaaccccgacctgcactatgcgatgacctttccggacggcacatacacactgtttgttccggcagggacccacacactgcaggccaaggcaggcggctacgtagcagccagccggcaggcgatctcgccggggacgggcgcagacttcaacctggcccaggacctgagcaccaagtacaccatcgacggcaacctggccgactggacggcccccaaggtgacgctgcaaagcccgaccgagggaggcttcgggcccgacaacaattggttgacactccaggccgacagtgatgaccactatctgtacctcgcgtacacgtaccgggtgaagggaaacagcgcgatcctgtacctggacaccaagatgggcggtgcggcccaagccgacaatttcgaggcttggaagcgggcggcgaccttcagtgggagcatggggggcgccgacgcctttgttgcgcggtacgaaaaccagatggctcaactgaggctgtttcagagcgatactgccacgcccgaggtcaacacgggcgactacaagtttgcagcgagcggtaccctgcccgagcagacggtggaactggcgatcccgtggacagcactcggcctcagcgaaaaacctgcgaacggtgtgaacgtggtgggtggaattttcggtggcgacggctacggcgcgggcgacatcgtgcccaataccaccagtacaccccccggtgccaacaccattggaacggatgccgaacagcgccgggcaaccttcactcagcccctcaacgtgaggtaa

SEQ ID NO: 4 - protein, 1021 aa, Deinococcus sp.
MKRFQKVGRSGALAVLTLALSACGVLKAPETGGNTRAWQDEVIYFAMTDRFANGNPANDNGPNRNEGDRADRTNPLGWHGGDFAGLKAKIEEGYFKRMGFTALWISPVVLQVPAIEGPKTGPNAGKLFAGYHGYWAEDFFKVDPHFGTLDEYKSLIQTAHRNGIKVIQDIVVNHAGYGATLTKTNPDWFHTQAECDASTNKRVDCPLAGLPDFKQERPEVTTYLNDFVNSWRKETGIDGLRIDTMQHVSDSYWQQFFAAGGPGDPSKIWSVGEVFNGDPAFLAHYMDDLGSPSVFDFALYFAIKDGLSSARGDLGRLADVFARDGAYRDPTRLTTFVDNHDVPRFVSEVQERGGTAAQANERLDLALSLIYTSRGTPSVYQGTEIAQPGLGDPYNYATGQGNREDMNFGALSQSSIDERLAALAAARAKYRALTHGVQQELWRPNGGAPIFAHRRIVTDGQGGQPVVVVINNGDTPVDLSTLSGGGIPLLGTFSGTALTEITGRTSDLSVSGGQLVGTVPARSALAVTAPAGSGSTGTVNPRLPEVTDLSAKAGDSAVQLTWTASTDLNVTGCRVYARTGSGQERLLNFAPLPKDQTTYLAAGIPNDQETTFRVVTVDAQGAESRGVSVKATPSSKNTVRVTFTVDARSQGNGPIELRRFDTGSQLEYPMTQVSRGIWKTAIDLPLFREIKFKFGNDGPAAKNSGYEAPGQPDRSYVVGTNPNVYTGTYDFITQPVPQTTIEGQVRGAGNPLANALVEAVTANPDLHYAMTFPDGTYTLFVPAGTHTLQAKAGGYVAASRQAISPGTGADFNLAQDLSTKYTIDGNLADWTAPKVTLQSPTEGGFGPDNNWLTLQADSDDHYLYLAYTYRVKGNSAILYLDTKMGGAAQADNFEAWKRAATFSGSMGGADAFVARYENQMAQLRLFQSDTATPEVNTGDYKFAASGTLPEQTVELAIPWTALGLSEKPANGVNVVGGIFGGDGYGAGDIVPNTTSTPPGANTIGTDAEQRRATFTQPLNVR

SEQ ID NO: 5 - DNA, 1545 bp, Deinococcus sp.
  CDS (1)..(1545)
atgcgccgcctgcccctcctcgccgccctgctcgcctcgctggccggcgcgcaggccaaaaaagcccagattcttcttgacacccacagaaccatcagcgaaatcagccactacatctttggtggattcgccgagcacatgggccgctgcatctacgagggcatctacgacccccaaagccctctgagcgacgagaacggcatccgcagggatgtgatggacgccctgaaggaactcaatttccgttccatccgttaccccgggggcaacttcgtgtcagggtacaactgggaagacggaattggccccagggaaaaccgcccggtcaagcgcgatctggcctggaggagcatcgaaaccaaccagtttggcacggatgaattcatgaaggtctgcgctgaactgaagaccgaacccatgatggccgtgaacctgggcaccggaagtattcaggacgcggccaacatcgtcgaatactgcaacctcgaaggcggcacccattacagcgacctgcgcatcaaaaacggtgctgaaaaaccttatggtgtgaagttctggtgtctggggaacgagatggatggtccctggcaggtgggacagctttctgcagaggattacagtaagaaagccgtgcaggctgcaaaggccatgaagctgatcgatccttccattcaactgattgcctgcggttcctcctccagcctcatgaactcctaccccgagtgggaccgcatcgtgctggaagagacctgggaccagatcgattacctctcgatgcactactatgccagcaaccgggaggaggacactgccagttacctcgcctatacccgtgaattcgaagaccacctgcaaaccctggccgccaccatccgttacgtgaaagccaagaaacgcagccagaaagacgtgttcctctcctgggatgaatggaacgtctggtaccgcgaaatgaacggcaacggcgagtggcagcaggccccccacatcctggaagaggtctacaaccttgaagatgcgctggtggtggcccagtggatgaatgtcctcctgaagcacagcaatgtgctgaagatggcctccatcgcacaggttgtcaatgtgatcgctcccatcatgaccagacgggatggcatgttcaaacagaccatctattatcctttcctggtgttcagcaaacacgcttctggtcaggcgctcagcctgcatgtggcctccgaccagtacgagacgaaaaaacacggcctcgtgaacctgctcgatgccagtgccagttttgatgccagccagaacgaaggggctgtttttctggtcaaccgcagccaggatgaagaactcgaaaccgaaatcgtctttcagggccgtgttcccacttccgtgcgcgtggcccaccagcttgctggcagcgaccccaaagcccacaactccttcgaggagcctgaaaagctcaccctgcagacgattgaagcaggggagatcaaagacggcaaactcgtgctgaagcttcctgccctgtccttttcggcagtggtgctggactactaa

SEQ ID NO: 6 - protein, 514 aa, Deinococcus sp.
MRRLPLLAALLASLAGAQAKKAQILLDTHRTISEISHYIFGGFAEHMGRCIYEGIYDPQSPLSDENGIRRDVMDALKELNFRSIRYPGGNFVSGYNWEDGIGPRENRPVKRDLAWRSIETNQFGTDEFMKVCAELKTEPMMAVNLGTGSIQDAANIVEYCNLEGGTHYSDLRIKNGAEKPYGVKFWCLGNEMDGPWQVGQLSAEDYSKKAVQAAKAMKLIDPSIQLIACGSSSSLMNSYPEWDRIVLEETWDQIDYLSMHYYASNREEDTASYLAYTREFEDHLQTLAATIRYVKAKKRSQKDVFLSWDEWNVWYREMNGNGEWQQAPHILEEVYNLEDALVVAQWMNVLLKHSNVLKMASIAQVVNVIAPIMTRRDGMFKQTIYYPFLVFSKHASGQALSLHVASDQYETKKHGLVNLLDASASFDASQNEGAVFLVNRSQDEELETEIVFQGRVPTSVRVAHQLAGSDPKAHNSFEEPEKLTLQTIEAGEIKDGKLVLKLPALSFSAVVLDY

SEQ ID NO: 7 - DNA, 2418 bp, Deinococcus sp.
  CDS (1)..(2148)
atgtgcctggagtgctttttcagggtcctccaggcgttgaaggccacacctgtcctcctgaggggcccttgttgcaccttcaggaggttcaatcccatgatgccaaccccttcaaaagtcctgcctgccagtgtgctgctgatggtctccctgctgaccagctcctgtaatctgttccagccacctgcacccaactgcacccccaagaccaccggagccaccgttccagcaggggactacgatcctgcagcaagtgaaaaagccttcccggacctgctgaccacagctgcccgcaaaccctcacaggccggagccctgcaactcgtgcagcaagactgcatggttacgctggcagacagcagcggcaaacccatccagcttcgtggcatgagcacccacggcctgcaatggtacccggaaatcgtcaatgacaatgccttcaaggccctggccaacgactgggggtccaatgtcttccgtctggccctgtacgtcggagaagggggatacgccaccaaacccgaactgaaacaaaaagtcattgaaggcattgattttgccattgccaacgacatgtacgtgattgtggactggcacgtgcatgcccctggcgaccccaacgcagacgtgtacaccaatgccaaaccgctggagttcttcaagtccatcgcccagaagtaccccaacaacaagcacatcatttacgaggtcgccaacgaacccaaccccggtcaggctccaggggtcagcaatgacgctgaaggctggaagaagatcaagtcctacgcagagcccatcatcaagatgctgcgggacctgggcaacaagaacatcgtgattgtcgggacccccaactggagccagcgcccggatctggctgccgacaaccccatcaaagacagtgccaccctttacactgtgcacttctacaccggcacccacatgccctccaccaacctggcagaccggggcaacgtgatgagcaatgcccgttacgccctggagcacggtgtgggcgtgttctccaccgagtggggggtcagcgaggccagcggaaacaacggacctttcctcaaagaagccgacgtctggctgcagttcctcaacaaacacaacatcagctgggtcaactggtctctcaccaacaaggccgagacttctgcagccttcctgccgttccccaaccagaccagccttgatcccggggcagacaggctgtggacccccagcgagctcaccctgtccggggaatacgttcgggcgcgcatcaaagggaccaaataccagcccattgaccgcactgccttcactgaggtggccttcaattttgacaacgacaccacccagggttttgccctgaacccggacagtggagtcaaagggatcacggtcagcgcagagaacaagatgctgaaactcagccccctgagcggaagcaatgacgtttcagcaggcaacttctgggccaacgcgcgcttctctgcagatggaaccagccagcatcccaacctgcggggtgcgaagagcatgagcatggatgtgtatgtgcctgcccccaccaaagtctccgtggccgctgtgccccagagcagcaaggatggctggaccaaccctgcgcgtgctgtgatcgtgaacgcagaccagtttgtgaaacaggcagatggcaagtacaaagccaccgtcaccctgtctgacgaagatgctcccaacctgaaactcattgccgaagatgagaccgacaatgtgctctcgaacctgatcctcttcattggcaccgagagccaggaagccaatgacaccgtgtggatcgacaacatcaccttctctggggaccgtgtggtggtccctgtggaacatgatcccatcggcaccgccaccctgccctccacatttgaagacagcacccgccagggctgggactgggccggagaatctggcgttaaaactgcactgaagatccagaccgccaatgcgtcaaaagccctgtcctgggatgtcatctaccctgatgtgaaacctgccgatggctgggcctctgctccccgtctggtgctggagaaatccaaccttacccgcggtgccaacaagtacctcgctttcgacctgtacctgaagccagatcgggccagcaaagggaccctctccgtcaacctggcttttggtcctccgagcctggggtactgggcacaggccagcgagaacgtcgacatcgacttgaccacgctgggggccatgaccaaaactgccgatggactgtaccgcattgcagggaaattcgatctggacaagatcaatgacaacaaggtgattgctgcagacactgttctgggcaaaatcaccctggtggtcgccgatgtgaacagcgattacgccgggaagatgttcctggacaacgtgcgtttcaccaacgaaccctaa

SEQ ID NO: 8 - protein, 716 aa, Deinococcus sp.
MCLECFFRVLQALKATPVLLRGPCCTFRRFNPMMPTPSKVLPASVLLMVSLLTSSCNLFQPPAPNCTPKTTGATVPAGDYDPAASEKAFPDLLTTAARKPSQAGALQLVQQDCMVTLADSSGKPIQLRGMSTHGLQWYPEIVNDNAFKALANDWGSNVFRLALYVGEGGYATKPELKQKVIEGIDFAIANDMYVIVDWHVHAPGDPNADVYTNAKPLEFFKSIAQKYPNNKHIIYEVANEPNPGQAPGVSNDAEGWKKIKSYAEPIIKMLRDLGNKNIVIVGTPNWSQRPDLAADNPIKDSATLYTVHFYTGTHMPSTNLADRGNVMSNARYALEHGVGVFSTEWGVSEASGNNGPFLKEADVWLQFLNKHNISWVNWSLTNKAETSAAFLPFPNQTSLDPGADRLWTPSELTLSGEYVRARIKGTKYQPIDRTAFTEVAFNFDNDTTQGFALNPDSGVKGITVSAENKMLKLSPLSGSNDVSAGNFWANARFSADGTSQHPNLRGAKSMSMDVYVPAPTKVSVAAVPQSSKDGWTNPARAVIVNADQFVKQADGKYKATVTLSDEDAPNLKLIAEDETDNVLSNLILFIGTESQEANDTVWIDNITFSGDRVVVPVEHDPIGTATLPSTFEDSTRQGWDWAGESGVKTALKIQTANASKALSWDVIYPDVKPADGWASAPRLVLEKSNLTRGANKYLAFDLYLKPDRASKGTLSV

SEQ ID NO: 9 - DNA, 2388 bp, Deinococcus sp.
  CDS (1)..(2388)
gtgaaactcggcgaggacacgaccgccccgtacgagttcacggtgaacgccgaccctggcctgaacggcacgcacgtctactccgcgcaggcggtggcgggcgacgcggccgggatctccgcgccggtcagcgtgcagatccgcatcgcggacacccgcaccaccgaactgctcagcaacggggacttcagccagggcctgaacccctggtggactgccggaacggccgccagcacgaccggcggtgagacctgcctgaacatcacgcagccgggcagcaacccctgggacgtgctgttcgggcagggcggcgtgggcctgaacgagggcggcacgtacaccctgagcttcacggcgcgcgccgcgcagcccacgtcgttcaggacgctgctgcagttcgacggcgcgccgtacaccaactacttcgtgcaggacgcggacgtgaccagccagccgaagaccttcacgtccacgttcacgatggcgcagcccagtgacgcgaaggccgcgttccagttccagctgggcgccagggccgccacgaccgtgtgcttcagccgcatttcactgactggccctgccttcggcagcgccgtgcccgcctcgggtgcggacgacctgaagctggtgcggctcaaccagaccgggtacctgccggaccggccgaaactggcggccctgccgttcgactcggaccggccgctgccgtggactctgctggacggcacgcgcacggtcgccagtggcgtgacccgcgtgttcggcgcggacgccgcgtccggcgagcacgtgcatcaggtggacttcagtgccgtgaccgccccggccgacgggctggtgctggacgtcgcgggtttccgcagccacccgttccggatcgggcgcgtgtacgacggcctgaaacgcgacgcgctggcgtacttctaccacaaccgcagcggcacgcccatcaaggcgaagtacgtcggggacgcctgggcgcgcccggccggtcacgccgggaccagcccgaaccagggggacacgcgcgtcagctgcttcaagggcaccgatcaggccgggaacgtctggcccggctgcgggtacgaactggacgccagcggcggctggtacgacgccggggatcacgggaagtacgtcgtgaacggcggcgtgagcgtctggacgctgctgaacctcgccgagcggggcgcgcgcctgaacctcccggacgcggacggcagcctgaatatcccggaaagcgggaacggccgcagcgacctgctggacgaggtccgctgggagctggacttcatgctgcggatgcaggtcccggatgggcagaccctggtcctgccgcgcggcgaccagcgcggcgccccgctgaccctgacgcccaccccggcgggcgggctggtgcaccagaaactcacggacgtcgcctggaccggcctgccgctgcgccccgatcaggatccgcagccgcgcgcgctgtactaccccacgaccgccgcgaccctgaacctcgcgggcgtggcggcgcagtgcgcccgcgtgtaccgcgcctcggacccggccttcgcggtccgctgcctgagcgccgcgcgccgcgcgtggcaggccgcgcgggccgcgccggacgtgtacgcgtacgacctgttcgtgggcggcggcccgtacgacgatacggacgtcagcgacgagttctactgggcggcggcggaactgtacgccacgaccggcgaggcggcgttcctggaggcgctgcgggccagtccgctgttcctgcagatgcccgaggggcgtgaactgggctggagtgacctgacggcagcgggcaccctgacgctcgccagcgtgcccacggcgctgcccggcgcggacgtgcagcaggcccgcgcgaacgtcgtggcggcggcgcgggcgttccgggacgcggcaggcacgcaggggtaccgcctgccgatgaccggcgccgaggccacctggggctcgaacagtggcgtgctgaaccgctcggtcgtgatgggcgccgcgtgggacttcacgggtgacgattcgttcgtgaatgtcgtgctggagggcctgaactacctgctgggacgcaacccgatggacaagtcgtacgtgtccgggtacggcgagcgtccgctgctgaacccgcaccaccgcttctgggcgcggtcgctggacgcggcgctgcccggcccgccgcgtggggtggtgtcgggcggcccgaacagcgtgaacttcagtgatccggtcgcggcgaaactcaggggccgctgcgtgggcctgcgctgctacaccgacgacatcggcgcgtacaccatgaacgaggtgaccatcaactggaacgcgccgctggcgtgggtggcggcgttcgtggagcacagcacccgacgctaa

SEQ ID NO: 10 - protein, 795 aa, Deinococcus sp.
VKLGEDTTAPYEFTVNADPGLNGTHVYSAQAVAGDAAGISAPVSVQIRIADTRTTELLSNGDFSQGLNPWWTAGTAASTTGGETCLNITQPGSNPWDVLFGQGGVGLNEGGTYTLSFTARAAQPTSFRTLLQFDGAPYTNYFVQDADVTSQPKTFTSTFTMAQPSDAKAAFQFQLGARAATTVCFSRISLTGPAFGSAVPASGADDLKLVRLNQTGYLPDRPKLAALPFDSDRPLPWTLLDGTRTVASGVTRVFGADAASGEHVHQVDFSAVTAPADGLVLDVAGFRSHPFRIGRVYDGLKRDALAYFYHNRSGTPIKAKYVGDAWARPAGHAGTSPNQGDTRVSCFKGTDQAGNVWPGCGYELDASGGWYDAGDHGKYVVNGGVSVWTLLNLAERGARLNLPDADGSLNIPESGNGRSDLLDEVRWELDFMLRMQVPDGQTLVLPRGDQRGAPLTLTPTPAGGLVHQKLTDVAWTGLPLRPDQDPQPRALYYPTTAATLNLAGVAAQCARVYRASDPAFAVRCLSAARRAWQAARAAPDVYAYDLFVGGGPYDDTDVSDEFYWAAAELYATTGEAAFLEALRASPLFLQMPEGRELGWSDLTAAGTLTLASVPTALPGADVQQARANVVAAARAFRDAAGTQGYRLPMTGAEATWGSNSGVLNRSVVMGAAWDFTGDDSFVNVVLEGLNYLLGRNPMDKSYVSGYGERPLLNPHHRFWARSLDAALPGPPRGVVSGGPNSVNFSDPVAAKLRGRCVGLRCYTDDIGAYTMNEVTINWNAPLAWVAAFVEHSTRR

SEQ ID NO: 11 - DNA, 1320 bp, Deinococcus sp.
  CDS (1)..(1320)
atgaaccctgtttcaggctttcttcaaagggttcttgcttttccagagcaaaaaagcacagcccctttcccagagcagaaagaaaggcatgagaaatgccattcatctgaacccggtacagccacagaatgcatgacagaaaaacattccatcgtgaaaaaaacgtcatacaataaaagcatgcatttccaacccagcgacgacagccaactgatcctttcccatgtgcgcgaattcgcacagcatgaaattgcccctctggccgcccagtacgaccgctctggagaatacccctgggagcaactgcgcaaactggccgacatgggccttctgggggccaccatccccgaggaatatggaggtgcaggtctggattcggtgacttacgccctctgtctggaagaaatcgctgctgccgatgccagtgtggccgtgatcatgagcgtccagaacggacttccagagcaaatgatcctgaagtacggctctgaagcacaaaaacagcagtacctcgctcctctggcgcagggcaaaaaaattggggccttctgtctcactgagccgaatgcaggttcggatgcagccagtttgcgtctgagtgctcaaaaagatggtgacagttgggttctgaacggccagaaagcatggatcacctctggaggacaggcggacacctatctggtgatggcccgcacctctggctctggggcaaaaggggtcacctgttttgtggtggaaaaagacacccccggcctctcttttggcaaacccgaggagaaaatgggccttcatgctgcccacaccaccacagtcaattttgaagagctgcggattccagatgcacaacgcattggcgatgaagggcaaggtctgatcatcgccctgagcagtctggattccgggcgcattggcattgccatgcagtccatcgggattgccagagcggctctggaagcagcagcaaagtacgccctgacccgcgagcaatttggcaagaaaattgcagagtttgagggggtctctttcaagattgcggaaatggcttcccgcattgaggctgcccgtctggtggccctcaaagctgcatggctgcgcgatcaggggaagaagtacaccaaagaggcttccatggccaagtttctggcttcggatgctgcggtgtacgtgacccgtgaagcggtgcagatttttgggggcaacgggtacagccgggaatacccggtggaacggtattaccgggacgccaaaatcaccgaaatctatgaaggcaccaacgagatccagaaactggtgatttcccgtcaggtgttctcagagtat

SEQ ID NO: 12 - protein, 440 aa, Deinococcus sp.
MNPVSGFLQRVLAFPEQKSTAPFPEQKERHEKCHSSEPGTATECMTEKHSIVKKTSYNKSMHFQPSDDSQLILSHVREFAQHEIAPLAAQYDRSGEYPWEQLRKLADMGLLGATIPEEYGGAGLDSVTYALCLEEIAAADASVAVIMSVQNGLPEQMILKYGSEAQKQQYLAPLAQGKKIGAFCLTEPNAGSDAASLRLSAQKDGDSWVLNGQKAWITSGGQADTYLVMARTSGSGAKGVTCFVVEKDTPGLSFGKPEEKMGLHAAHTTTVNFEELRIPDAQRIGDEGQGLIIALSSLDSGRIGIAMQSIGIARAALEAAAKYALTREQFGKKIAEFEGVSFKIAEMASRIEAARLVALKAAWLRDQGKKYTKEASMAKFLASDAAVYVTREAVQIFGGNGYSREYPVERYYRDAKITEIYEGTNEIQKLVISRQVFSEY

SEQ ID NO: 13 - DNA, 1026 bp, Deinococcus sp.
  CDS (1)..(1026)
atgcaagctgttgcactcaccagacggggcaacattgacgcccttgaacccatccgcctgcccatttcagagcctcaagctggagaagtcctggtgcgcatccgtgcagtggccctcaaccacctggatgtgtgggtgcgcaagggggtcgccagcccgaaacttcccctgccacacctgctcggctcagacattgcgggagaggtggctgcaatgggtccaggggttgaaggtttgtcagagggcacaaaagtgatgctgaacccgggcgtatcctgcggccactgcgaacgctgcctcagtggacacgacaacctgtgccgccactaccaaattctcggggaacaccgctggggagggtatgcccagtacatcagcatcccgagaaccaatgtgctgcccatgccagagggccttgactttgtagaggccgcctctgtccccctgtcggccctcaccgcttaccagatggtgttcgaccgtgcacagctgaaaccctgggaaaccgtcctgatcctggcggcggcaagtggggtcagcgtcaacctgatccagctctgcaaactggtgggtgcaaaagtcatcgctgtggccagcacccctgaaaagcaggccctggcactgaaacttggtgcagatcacgtgatcggttcacatgaagaccaggctcaggccgtcaaagccctgactgcaggagaaggggcagacgtggtgtttgaccacaccggagcggacaactggcaacgcagcctgaaaagcctgaagtggggaggccgtctggtcacctgtggggcaaccagtggacatgaagccgtgacccccctcaactgggtgtttttcaagcagctcagcatcctgggttccaccatgggctccaaagcagacctccacaaaattcagaagtttgtgcaagaaggaaaactcaggcctgtggtgggccatgttctggactttgctcaggcaagagaggcccatgggcttctggaatccaggcaggctctgggcaaggtggttttgagggtcccg

SEQ ID NO: 14 - protein, 342 aa, Deinococcus sp.
MQAVALTRRGNIDALEPIRLPISEPQAGEVLVRIRAVALNHLDVWVRKGVASPKLPLPHLLGSDIAGEVAAMGPGVEGLSEGTKVMLNPGVSCGHCERCLSGHDNLCRHYQILGEHRWGGYAQYISIPRTNVLPMPEGLDFVEAASVPLSALTAYQMVFDRAQLKPWETVLILAAASGVSVNLIQLCKLVGAKVIAVASTPEKQALALKLGADHVIGSHEDQAQAVKALTAGEGADVVFDHTGADNWQRSLKSLKWGGRLVTCGATSGHEAVTPLNWVFFKQLSILGSTMGSKADLHKIQKFVQEGKLRPVVGHVLDFAQAREAHGLLESRQALGKVVLRVP

SEQ ID NO: 15 - DNA, 1710 bp, Zymomonas mobilis
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgccgctgaagcgatttacaccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggcacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcattcgcttccccagcgtccatctgaaagactatctgacccgtttggctcagaaagtttccaagaaaaccggtgcattggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagtag

SEQ ID NO: 16 - DNA, 1707 bp, ARTIFICIAL
  Deinococcus codon optimized PDC gene derived from Zm
atgagctacacggtgggcacgtacctggcggagcggctggtgcagatcggcctgaagcaccactttgcggtggcgggcgactacaacctggtgctgctggacaacctgctgctgaacaagaacatggagcaggtgtactgctgcaacgagctgaactgcggctttagcgccgagggctacgcccgtgccaagggcgctgccgccgccgtggtgacctacagcgtgggcgccctgagcgcgttcgacgccatcggcggcgcctacgcggagaacctgccggtgatcctgatcagcggtgcccccaacaacaacgaccacgccgcgggccacgtgctgcaccacgccctgggcaagaccgactaccactaccagctggagatggcgaagaacatcaccgctgccgccgaggccatctacacgccggaggaggcccccgcgaagatcgaccacgtgatcaagaccgccctgcgcgagaagaagccggtgtacctggagatcgcgtgcaacatcgccagcatgccctgcgcggcccccggcccggccagcgcgctgttcaacgacgaggcgagcgacgaggccagcctgaacgcggccgtggaggagaccctgaagtttatcgcgaaccgcgacaaggtggccgtgctggtgggcagcaagctgcgggcggccggcgccgaggaggcggccgtgaagttcgccgacgcgctgggcggcgccgtggcgacgatggccgccgccaagagcttctttccggaggagaacccccactacatcggcaccagctggggcgaagtgagctacccgggcgtggagaagacgatgaaggaggccgacgcggtgatcgccctggcgcccgtgtttaacgactacagcaccacgggctggaccgacatccccgacccgaagaagctggtgctggccgagccgcgcagcgtggtggtgaacggcgtgcggttccccagcgtgcacctgaaggactacctgacccgcctggcgcagaaggtgagcaagaagacgggcgccctggacttctttaagagcctgaacgcgggcgagctgaagaaggccgcgcccgcggacccgagcgcccccctggtgaacgcggagatcgcccggcaggtggaggccctgctgaccccgaacaccacggtgatcgccgagacgggcgacagctggttcaacgcgcagcgcatgaagctgcccaacggcgcccgggtggagtacgagatgcagtggggccacatcggctggagcgtgccggccgcgttcggctacgcggtgggtgcccccgagcgccggaacatcctgatggtgggcgacggcagctttcagctgaccgcgcaggaggtggcccagatggtgcgcctgaagctgccggtgatcatcttcctgatcaacaactacggctacacgatcgaggtgatgatccacgacggcccctacaacaacatcaagaactgggactacgccggcctgatggaggtgtttaacggcaacggcggctacgacagcggcgcgggcaagggcctgaaggccaagaccggcggcgagctggccgaggcgatcaaggtggccctggcgaacaccgacggccccacgctgatcgagtgcttcatcggccgggaggactgcacggaggagctggtgaagtggggcaagcgcgtggccgccgccaacagccgcaagccggtgaacaagctgctgtga

SEQ ID NO: 17 - DNA, 1152 bp, Zymomonas mobilis
atggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgctttgaaagcagcttccatgatcgctaagaatctgaagaccgcttgcgacaacggtaaggatatgccggctcgtgaagctatggcttatgcccaattcctcgctggtatggccttcaacaacgcttcgcttggttatgtccatgctatggctcaccagttgggcggttactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaacctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaa

SEQ ID NO: 18 - DNA, 1152 bp, ARTIFICIAL
  Deinococcus codon optimized ADH gene derived from Zm
atggcgagcagcacgttttacatcccgtttgtgaacgagatgggcgagggcagcctggagaaggcgatcaaggacctgaacggcagcggcttcaagaacgccctgatcgtgagcgacgcgtttatgaacaagagcggcgtggtgaagcaggtggccgacctgctgaaggcgcagggcatcaacagcgccgtgtacgacggcgtgatgcccaacccgaccgtgacggcggtgctggagggcctgaagatcctgaaggacaacaacagcgacttcgtgatcagcctgggcggcggcagcccgcacgactgcgccaaggcgatcgccctggtggccaccaacggcggcgaggtgaaggactacgagggcatcgacaagagcaagaagcccgccctgccgctgatgagcatcaacaccacggccggcacggcgagcgagatgacccgcttctgcatcatcacggacgaagtgcggcacgtgaagatggccatcgtggaccgccacgtgaccccgatggtgtccgtgaacgacccgctgctgatggtgggcatgcccaagggcctgaccgccgcgacgggcatggacgccctgacccacgcctttgaggcgtacagcagcaccgccgcgacgccgatcaccgacgcgtgcgccctgaaggccgcgagcatgatcgccaagaacctgaagaccgcgtgcgacaacggcaaggacatgcccgcccgggaggcgatggcctacgcgcagttcctggccggcatggcgtttaacaacgcgagcctgggctacgtgcacgcgatggcccaccagctgggcggctactacaacctgccgcacggcgtgtgcaacgccgtgctgctgccccacgtgctggcctacaacgcgagcgtggtggcgggccggctgaaggacgtgggcgtggcgatgggcctggacatcgcgaacctgggcgacaaggagggcgccgaggcgacgatccaggccgtgcgcgacctggccgcgcccatcggcatcccggccaacctgaccgagctgggcgcgaagaaggaggacgtgcccctgctggccgaccacgcgctgaaggacgcctgcgcgctgaccaacccccggcagggcgaccagaaggaggtggaggagctgtttctgagcgcgttttga

SEQ ID NO: 19 - DNA, 1200 bp, Moorella sp
atgtgggaaacaaaaataaacatcaacgaagtccgggaaatccgggctaaaacaaccgtctactttggagttggagctattaaaaagattgacgacatagccagggaatttaaggaaaagggatacgataggatcatcgtaataaccggcaagggggcttataaagccaccggcgcgtgggaatatatagttccggccttaaataaaaaccagataacctatatccattacgaccaggtgacgcccaacccgacggtagaccaggttgacgaggcaaccaaacaagcccgggaattcggtgcccgagccgtcctggccatcggcgggggtagccccattgatgccgctaaaagcgtagccgtcttgctctcctaccccgacaaaaatgcccgacagctctaccagttagaatttacacctgttaaggccgcacctatcatcgctattaatcttacccatggtacggggacggaagccgatcgctttgccgttgtcagcatccctgaaaaggcatataaacccgctattgcctatgattgcatttaccccttatattcaattgacgacccggccctcatggtaaaactgccgtccgaccagacagcttatgtctctgttgatgccctcaaccatgtcgtcgaagcagccaccagcaaagtagccagcccctatactattatcctggccaaggaaacggtacggctcatcgcccgatacctgccccaggccctgtcccatccggcggatttgacggccaggtattatctcctctatgcttccctgattgccggaatagcctttgacaacggtttgctccacttcacccacgccctggaacaccccctgagcgccgtcaaaccggagctcgcccacggtctggggctgggtatgctgctgccggccgtagtcaagcagatttacccggcaaccccggaggtactggcggagatactggagcccattgttcccgatctcaaaggcgttcccggtgaagcagaaaaggctgccagcggggtggcaaaatggcttgccggagccggtattaccatgaagctaaaagatgcgggctttcaagcggaagatatcgccaggttaactgacctggcctttaccaccccgagtctcgagcttctcctgagtatggccccggtaacggccgacagggaaagggttaaggcaatttaccaggacgccttttaa

SEQ ID NO: 20 - DNA, 1532 bp, Zymomonas mobilis
atgaaaacaaaacatctttctgacagtattgtcgtaacatttataagcacttattctgattaaacatacgaaaattgctattgtgagtcaaatatcacaccttaagttacaaacttatagttgtacacgtttggtctaaaaaaaccttttatgtggcttttactagatacgaatgagcacatttggagggttcgctatggtgtgggaatcccacgttccgataaaccaggtctttgagctgaggtgcaaaacaacagactacttcggcctatgcgccatacacaaatttaacggcttcgtccgggagcttaaggggaaaggcgtggacagggttatcctcgttactgggagcagctcctacaagaagtgcggtgcatgggacgttgtcaggcctgcccttgaggaaaacggggtcgagtatgtccattacgacaaggtgggggccaacccaacggttgacatgatcgatgaagctgccgagatggggagagagttcggggcccaagcggtgataggcataggtggtggcagccccatcgatagtgccaagagcgttgcgattctgctggaataccctgacaagaccgccagggacctctacgagttcaggtttacccccgtaaaggccaagccagtaatagcgataaacacaacccatggagccggaaccgaagttgacaggttcgcagtggcgacgattcctgagaaagagtacaagccggccatagcttacgactgcatctacccgctttacgcaatcgatgacccctcactgacggtaaagctttccccggagcagaccctttacctgaccatcgatgcactcaatcacgttaccgaagccgccacaaccaagctggccaatccatattcgatactcctcgccaaagaggccgcgaggctgatatttgactacctcccagaggccctcaaacaccccgacaaccttcaggcaaggtacgccctgctctacgcctctgccatagccggaataagcttcgacaacggtctgctccactttacgcacgcccttgagcacccgctgagtgcggtcaaaccggacttcccccacggcattggcctcgcaatgcttcttccggcggtaatcaggcacatataccctgccaccgcaaagatacttgccgaggtctacaggccgctcgttcccgaggccaaaggtgttccgggagaggtggaactcgtcgccaggagggtagaagagtggctcttcagcatcggcataactgaaaagcttgaggacgtcgggttctctgagacggatgtgaacaggctaacggaacttgccatgaaaacaccaagccttaacctgctcctctccatggctccgggtgaggccactcgggagagaatagcggccatataccgtgattcgctttatcccatcagcagagggtgaggggcgctttcttctgccttttgattttctgtccgctttgcgaggattttaaacccttggttcaaaaaatctttttattggcttacttttagaccggatttgagaaagactctaa

SEQ ID NO: 21 - DNA, 417 bp, ARTIFICIAL
  pTufA promoter derived from Deinococcus radiodurans R1
gtcgggtgtcgagcatcgtgatcgctgtgctgagcctgctgttttttccctttggaacggtgctcggtgtcatcatgttgatcgggatcttcgacgaacaggtgaccgcttacctgagccgctgagcgggttttcgggcaagagttagttcggggccaggaacctatacggaacctatacgggaactggccctttttgctggattcccggcccggcgctcttgcccttctaggccttttcatgtaagctaagcagtcggtgcgccgtggcctctcagcccggtgcaacgtgccagagagcaggcgggacaccgccctggcgagaatcaacccaatgtgggtcacaccaccagaacctcgcggcatgcgcgggggaagcccgatcaaagggacgtttttacgtgtgaatccaccgctt

SEQ ID NO: 22 - DNA, 220 bp, ARTIFICIAL
  pTufB promoter derived from Deinococcus radiodurans R1
gttcgagtctcgtttcccgctttttacgctcctgtagctcagcggtagagcactcccttggtaagggagaggtcatcggttcaagtccgatcaggagctccaacgaagaaatgcggcccggataagagggccgcatttcgattttgaccgagcatggccctacgccagcccgcgcacttttgatatagtgttcaggcttgcccgtttgcgggcaaaaccc

SEQ ID NO: 23 - DNA, 651 bp, ARTIFICIAL
  Deinococcus codon optimized CATgene derived from Staphilococcus aureus
atgaactttaacaagatcgacctggacaactggaagcggaaggagatctttaaccactacctgaaccagcagacgacgtttagcatcaccacggagatcgacatcagcgtgctgtaccgcaacatcaagcaggaaggctacaagttctaccccgccttcatctttctggtgacccgcgtgatcaacagcaacaccgcgtttcggacgggctacaacagcgacggcgagctgggctactgggacaagctggagcccctgtacacgatcttcgacggcgtgagcaagacctttagcggcatctggacgccggtgaagaacgacttcaaggagttttacgacctgtacctgagcgacgtggagaagtacaacggcagcggcaagctgttccccaagacccccatcccggagaacgcctttagcctgagcatcatcccgtggaccagcttcacgggctttaacctgaacatcaacaacaacagcaactacctgctgcccatcatcaccgccggcaagttcatcaacaagggcaacagcatctacctgccgctgagcctgcaggtgcaccacagcgtgtgcgacggctaccacgcgggcctgtttatgaacagcatccaggagctgagcgaccgcccgaacgactggctgctgtga

SEQ ID NO: 24 - DNA, 375 bp, ARTIFICIAL
  Deinococcus codon optimized BLEO gene derived from Streptoalloteichus hindustanus
atggcgaagctgacgagcgcggtgcccgtgctgacggcgcgggacgtggcggaggcggtggagttctggacggaccggctgggctttagccgcgtgttcgtggaggacgactttgccggcgtggtgcgggacgacgtgaccctgttcatcagcgcggtgcaggaccaggtggtgcccgacaacacccaggcgtgggtgtgggtgcgcggcctggacgagctgtacgcggagtggagcgaggtcgtgagcaccaactttcgcgacgccagcggcccggccatgaccgagatcgtggagcagccgtggggccgggagttcgcgctgcgggacccggcgggcaactgcgtgcactttgtggcggaggagcaggactga

SEQ ID NO: 25 - DNA, 23 bp, ARTIFICIAL
  primer
ggtatctacgcattccaccgcta

SEQ ID NO: 26 - DNA, 23 bp, ARTIFICIAL
  primer
gttacccggaatcactgggcgta

(19) United States
(12) Patent Application Publication
(10) Pub. No.: US 2014/0356923 A1
(43) Pub. Date: Dec. 4, 2014

(54) BACTERIA WITH RECONSTRUCTED TRANSCRIPTIONAL UNITS AND THE USES THEREOF

(71) Applicant: DEINOVE, GRABELS (FR)

(72) Inventor: Jean-Paul Leonetti, Montpellier (FR)

(21) Appl. No.: 14/366,314

(22) PCT Filed: Dec. 21, 2012
(86) PCT No.: PCT/EP2012/076571

§ 371 Date: Jun. 18, 2014

Related U.S. Application Data
(60) Provisional application No. 61/579,808, filed on Dec. 23, 2011.

(30) Foreign Application Priority Data
Dec. 23, 2011 (EP) 11306767.2

(51) Int. Cl. C12P 7/10; C12N 9/42; C12N 9/24; C12N 9/28

(52) U.S. Cl. CPC C12P 7/10; C12N 9/2417; C12N 9/2437; C12N 9/2402; C12P 2203/00; C12P 2201/00
USPC 435/161; 435/252.3; 435/202

(57) ABSTRACT

The present invention relates to recombinant bacteria and the uses thereof, particularly for the production of ethanol. The invention also relates to methods for the production of such bacteria, as well as to nucleic acid constructs suitable for such production. The invention specifically relates to bacteria having a reconstructed biomass degradation unit.

The present invention relates to recombinant bacteria, their preparation, and the uses thereof. More particularly, the invention relates to bacteria having reconstructed transcriptional units and their uses for the conversion of biomass and/or the production of biofuel, particularly ethanol. The invention also relates to nucleic acid constructs, mixed cultures, compositions of bacteria or isolated extracts thereof, as well as methods of producing bioethanol.

### INTRODUCTION

Biofuels may be produced from biomass material through a number of process steps, including biomass degradation and fermentation, using e.g., chemical, physical and/or biological treatments and catalysts. Typically, biofuel production requires pretreatment of the biomass to at least partially hydrolyze the hemicellulose, remove the lignin and de-crystallize the cellulose, so that cellulase enzymes can access their substrate. Furthermore, in order to efficiently convert sugars into ethanol, microorganisms should exhibit specific properties such as high ethanol yield and productivity, high tolerance to acids, ethanol and inhibitors, and be active in simple growth medium and under wild process conditions. Production of biofuels such as bioethanol from lignocellulose would have the further advantage of abundant, diverse, and low cost raw material. However, this also requires a substantial amount of processing to make the sugars available to fermentation by microorganisms that are typically used to produce ethanol.

No natural microorganism, including bacteria or yeasts, meets all of these requirements.

Since the last decades, microorganisms have been selected or manipulated in order to improve their performance for production of ethanol. Gram-negative bacteria, such as Escherichia coli, Klebsiella oxytoca, and Zymomonas mobilis, Gram-positive bacteria such as Clostridium cellulolyticum or Lactobacillus casei, and several yeast strains have been engineered for bioethanol production from cellulosic substrates. Particularly, biosynthetic genes (such as PDC or ADH genes) have been cloned into bacterial strains. Also, competing pathways have been altered.

These microorganisms still show drawbacks. In particular, potential ethanol-producing microorganisms such as Zymomonas mobilis and Saccharomyces cerevisiae are typically not able to hydrolyze complex sugars such as lignocellulose. Z. mobilis not well suited for biomass conversion because it ferments only glucose, fructose and sucrose. Also, it shows very low tolerance to acetic acid. For Saccharomyces yeasts, optimal temperature is often around 37° C., which may not be optimal in large industrial culture facilities where the temperature can increase substantially.

Genetically altered gram-positive or Geobacillus strains have been mentioned (see WO95/27064 and WO2006/131734). From the industrial perspective however, no satisfactory metabolite production has been disclosed for these strains. Furthermore, Geobacillus strains produce spores, which is a substantial drawback for industrial use.

Obtaining high ethanol yields means finding strains that produce ethanol with few side products and metabolize all major sugars such as glucose, xylose, arabinose, galactose and mannose. Also, proper industrial process would require that enzyme and culture conditions be compatible with respect to pH and temperature.

Deinococcus is a gram positive bacterium that was isolated in 1956 by Anderson and collaborators. This extremophile organism is resistant to DNA damage by UV and ionizing radiations or by cross-linking agent (mitomycin C) and is tolerant to desiccation. WO01/023526 shows the unusual resistance of Deinococcus to radiation and further proposes their engineering and use in bioremediation. WO2009/063079 shows that Deinococcus bacteria can resist to solvents and transform biomass to generate ethanol. WO2010/130806 further discloses recombinant Deinococcus strains wherein ethanol biosynthesis genes have been inserted. These recombinant strains do exhibit improved performance in the production of ethanol.

The present invention now discloses a further generation of improved bacteria, with higher and remarkable biomass degradation properties. More particularly, the invention discloses engineered Deinococcus or related bacteria with improved biomass degradation and biofuel production properties. These bacteria have been engineered by the inventors to contain reconstructed functional transcriptional units and/or modified metabolic pathways, leading to substantially improved biological performances. Advantageously, these bacteria have been engineered with particular genes isolated and characterized by the inventors, or modified by the inventors to improve their expression, resulting in non-GMO, improved bacteria. These bacteria are compatible with industrial culture conditions, use raw biomass material, and have been engineered to maintain proper expression of integrated genes for optimal ethanol production. These bacteria, extracts thereof, or compositions comprising the same, are particularly suitable for use to modify biomass and produce biofuels.

### SUMMARY OF THE INVENTION

An object of the invention relates to Deinococcus or related bacteria comprising a reconstructed biomass degradation transcriptional unit inserted in their genome. More specifically, these bacteria comprise a reconstructed biomass degradation transcriptional unit comprising at least 2 genes under the control of a single promoter, said at least 2 genes encoding distinct biomass degradation enzymes.

In a preferred embodiment, the reconstructed biomass degradation transcriptional unit does not contain non-Deinococcus genetic material, which improves their activity and increases regulatory acceptance.

In another preferred embodiment of the invention, the bacteria further comprise a recombinant alcohol production transcriptional unit, preferably a recombinant ethanol production transcriptional unit.

In a further preferred embodiment, the bacteria contain altered ethanol-competing biosynthetic pathways. More specifically, particular objects of this invention resides in Deinococcus or related bacteria comprising an inactivated endogenous gene selected from a phosphate acetyl transferase gene, an Alanine dehydrogenase gene, a glucose dehydrogenase gene, a phosphoenolpyruvate carboxykinase gene, a phosphoenolpyruvate carboxylase gene, and/or a malate dehydrogenase gene.

A further object of the invention relates to a composition comprising a bacterium as defined above and at least one other bacterium.

A further object of the invention relates to a composition comprising a bacterium as defined above and a culture medium.

A further object of the invention relates to an enzymatic extract of a bacterium as defined above.

The invention also concerns a biotacalyst comprising a bacterium or an extract thereof as defined above.

The invention further resides in a process for transforming biomass, comprising exposing a biomass to a bacterium, or an extract, or a composition as defined above.

A further object of the invention is a process for producing an alcohol, particularly ethanol, comprising exposing a sugar or biomass to a bacterium, or an extract, or a composition as defined above and, preferably, collecting the alcohol produced.

The invention also relates to the use of a bacterium as defined above for producing an alcohol, particularly ethanol.

The invention also relates to a method for producing a Deinococcus or related bacterium, or an ancestor thereof, the method comprising:

a) providing a parent Deinococcus or related bacterium;
b) simultaneously or sequentially inserting into the genome of said parent bacterium at least 2 genes under the control of a single promoter, said at least 2 genes encoding distinct biomass degradation enzymes, and
c) selecting a bacterium of b) which expresses both said at least 2 genes.

The invention also relates to a nucleic acid comprising a sequence selected from SEQ ID NOs: 11, 13, 16, 18, 23, and 24.

The invention also relates to an isolated protein comprising an amino acid sequence selected from SEQ ID NOs: 12 and 14, or a functional variant or fragment thereof.

### LEGEND TO THE FIGURES

FIG. 1: Schematic representation of insertion steps to build Deinococcus with reconstructed biomass degradation unit.

FIG. 2: Amylolytic activity of reconstructed cells.

FIG. 3: Amylase activity of reconstructed cells. Cultivation was carried out in two parallel 5 L shake flasks containing 2 L of medium. On the left: OD600 measurements as a function of time. On the right: Ceralpha activities as a function of time measured from the cell free supernatant (blue and red) and cell pellets (violet and green). Activity was determined at 45° C. in 100 mM MOPS buffer (pH 7.0) containing 1 mM CaCl₂.

FIG. 4: Localization of the α-amylase activities in the wild M23-3A strain and the reconstructed cells. Concentrated (20×) culture supernatants (2=M23-3A, 3=DG_{—}4), cell pellets (4=M23-3A, 5=DG_{—}4) and Triton X-100 extracts of the cell pellets (6=M23-3A, 7=DG_{—}4) were analyzed by SDS-PAGE and by the zymogram technique (pH 7.0). SDS-PAGE was performed in non-reducing conditions without boiling the samples.

FIG. 5: Biomass and Glucose quantification during the cultivation on whole wheat 3%.

FIG. 6: Ethanol and organic acids quantification during the cultivation on whole wheat 3%.

FIG. 7: Biomass and sugars quantification during the cultivation on starch milk 20%.

FIG. 8: Ethanol and organic acids quantification during the cultivation on starch milk 20%.

### DETAILED DESCRIPTION OF THE INVENTION

The present invention relates to Deinococcus or related bacteria and the uses thereof for transforming biomass and/or producing biofuel or other metabolites.

The present disclosure will be best understood by reference to the following definitions:

### DEFINITIONS

Within the context of the invention, the term “derived from a bacterium” in relation to an enzyme indicates that the enzyme has been isolated from such a bacterium, or that the enzyme comprises all or a biologically active part of the amino acid sequence of an enzyme isolated or characterized from such a bacterium. The term “derived from a Deinococcus bacterium or related bacterium” further includes any recombinant, synthetic and/or optionally modified enzyme (e.g., modified chemically, enzymatically, physically) synthesized from a nucleic acid or amino acid sequence identified in a Deinococcus or a related bacterium.

Deinococcus bacteria designate any bacterium of the genus Deinococcus, such as without limitation, a D. geothermalis, D. cellulolysiticus, D. radiodurans, D. proteolyticus, D. radiopugnans, D. radiophilus, D. grandis, D. indicus, D. frigens, D. saxicola, D. maricopensis, D. marmoris, D. deserti, D. murrayi, D. aerius, D. aerolatus, D. aerophilus, D. aetherius, D. alpinitundrae, D. altitudinis, D. apachensis, D. aquaticus, D. aquatilis, D. aquiradiocola, D. aquivivus, D. caeni, D. claudionis, D. ficus, D. gobiensis, D. hohokamensis, D. hopiensis, D. misasensis, D. navajonensis, D. papagonensis, D. peraridilitoris, D. pimensis, D. piscis, D. radiomollis, D. roseus, D. sonorensis, D. wulumuqiensis, D. xibeiensis, D. xinjiangensis, D. yavapaiensis or D. yunweiensis bacterium. Preferred Deinococcus bacteria are D. geothermalis, D. cellulolysiticus, D. deserti, D. murrayi, and D. radiodurans.

A bacterium “related” to Deinococcus designates a bacterium which (i) contains a 16S rDNA which, upon amplification using primers GTTACCCGGAATCACTGGGCGTA (SEQ ID NO: 26) and GGTATCTACGCATTCCACCGCTA (SEQ ID NO: 25), generates a fragment of about 158 base pairs and/or (ii) resists a UV treatment of 4 mJ/cm². In a particular embodiment, Deinococcus-related bacteria are bacteria having a 16S rDNA molecule which is at least 70%, preferably at least 80% identical in sequence to a Deinococcus 16S rDNA sequence.

A “gene” designates any nucleic acid encoding a protein. The term gene encompasses DNA, such as cDNA or gDNA, as well as RNA. The gene may be first prepared by e.g., recombinant, enzymatic and/or chemical techniques, and subsequently replicated in a host cell or an in vitro system. The gene typically comprises an open reading frame encoding a desired protein. The gene may contain additional sequences such as a transcription terminator, a signal peptide, an IRES, an intron, etc. Preferably, the gene does not contain an intron.

A “transcriptional unit” designates, within the present invention, a group of at least one gene under the control of one promoter.

The term “reconstructed” or “recombinant” in relation to a sequence, nucleic acid, or unit in a bacterium, indicates the sequence, nucleic acid or unit does not exist naturally in the bacterium and has been assembled and/or inserted in said bacterium or an ancestor thereof. In a reconstructed or recombinant unit, the sequences are preferably of the same origin as the bacterium in which they are assembled or inserted. As an example, a reconstructed unit in a Deinococcus bacterium preferably essentially comprises nucleic acid derived from Deinococcus bacteria.

The term “fragment”, in relation to a protein or enzyme, designates any fragment of thereof comprising at least about 10, 15, 20, 25, 40, 50 or even more preferably at least 60 contiguous amino acids of said protein. Most preferred fragments are functional, either by themselves or when fused to or combined with another polypeptide. A fragment of a protein also designates a mature form of the protein (i.e., that does not contain a signal peptide at the N-terminal end of the protein).

The term “variant”, in relation to a protein or enzyme, designates any protein that exhibits at least 50% amino acid sequence identity to the reference protein, even more preferably at least 60%, 70%, 80% or 90%, and that retains an activity of the reference protein. The extent of sequence identity (homology) may be determined using any computer program and associated parameters, including BLAST 2.2.2 or FASTA version 3.0t78, with the default parameters. Preferred variants have a level of identity of at least 90% with the reference sequence, most preferably of at least 92, 95, or 97%. In a preferred embodiment, variants comprise at most between 1 to 50, 1 to 40, 1 to 30, 1 to 25, 1 to 20, 1 to 15, 1 to 10, or 1 to 5 modified (e.g., deleted, substituted or inserted) amino acid residues as compared to the reference protein. Proteins qualify as variants if they exhibit at least 20%, preferably at least 30% and more preferably at least 50% of an enzymatic activity of the reference protein.

The term “biomass” according to the invention typically designates a biomass comprising in particular cellulose and/or xylan. The term biomass thus includes organic material of biological origin, including vegetal or animal organic material. The biomass may be unprocessed or pre-treated. The biomass is typically a cellulose- or xylan-containing organic vegetal or animal material. Examples of biomass include, without limitation, forestry products, including mature trees unsuitable for lumber or paper production, organic waste, agricultural products, such as grasses, crops and animal manure, and aquatic products, such as algae and seaweed. Examples of biomass include wood or vegetal material derived from numerous types of plants, including miscanthus, hemp, sugarbeet, wheat, corn, poplar, willow, sorghum, sugarcane, and a variety of tree species, ranging from eucalyptus to oil palm. Specific sources of biomass include, without limitation, plant residues, hardwood or softwood stems, cobs, straw, grass, leaves, seeds, paper, etc. (see for instance Sun et al., Bioresource Technology 83 (2002) 1-11). The term biomass also encompasses transformed biomass or secondary biomass, which essentially contains hydrolysed pre-treated biomass products.

“Moding” a biomass within the context of the present invention includes any modification thereof, including transformation, degradation, hydrolysis, conversion or processing. The term “modifying” a biomass typically encompasses any modification of the biomass that results in the production of fermentable sugars. Modification also typically encompasses the hydrolysis of biological polymers of the biomass.

A “biomass degradation enzyme” is an enzyme that is involved in the degradation of a biomass (or of a component of a biomass) into degraded products. A biomass degradation enzyme is preferably an enzyme that contributes to the degradation or hydrolysis of biomass into fermentable sugars. Examples of such enzymes include amylases, cellulases, arabinofuranosidases (such as e.g., alpha-L-arabinofuranosidases), xylanases, laccases, alpha-glucuronidases, and esterases, such as ferulic acide esterases or acetyl xylan esterases.

The term “alcohol” or “bioalcohol” more specifically designates a linear or branched alcohol, diol or triol comprising from 1 and 5 carbon atoms, preferably from 1 to 4 carbon atoms. Specific and preferred examples of “alcohols” include C₁₋₄ alcohols selected from methanol, ethanol, propanol, isopropanol, propanediol, butanol, 2,3-butanediol, 1,4-butanediol, isobutanol, or glycerol, more preferably ethanol.

The term “biofuel” according to the invention includes, without limitation, vegetable oils, biodiesels, bioalcohols, biogas, syngas and solid biofuels.

### Biomass Degradation Transcriptional Unit

Deinococcus bacteria have been shown to have the capacity to reassemble their genome, in full or in part, when disrupted by a stress. The ability of Deinococcus bacteria to produce bioenergy products from biomass is disclosed in WO2009/063079. The present invention now shows that the performance of these bacteria can be improved by re-engineering metabolic pathways. More particularly, the invention provides novel bacteria having a reconstructed biomass degradation transcriptional unit. The invention shows such a unit can be inserted in the genome of a Deinococcus or related bacterium without altering cellular growth. The invention shows that expression of several genes from a single promoter is feasible and allows a better regulation of the levels of enzymes in the cell. The invention further describes bacteria comprising a functional reconstructed biomass degradation unit with 5 genes in a single insertion site, which exhibits improved performances. The invention further describes such re-engineered bacteria constructed with Deinococcus-derived nucleic acid, and which do not contain heterologous genetic material.

These bacteria represent valuable products and biocatalysts and are particularly adapted to modify biomass with improved capacity.

A biomass degradation transcription unit of the invention designates preferably a nucleic acid molecule which comprises at least two distinct genes placed under the control of a single promoter, said two distinct genes encoding two distinct biomass degradation enzymes. The present invention discloses the insertion, in one single location within the genome of a Deinococcus bacterium, of several genes under the control of a unique promoter. Such configuration provides optimal expression of the genes and does not affect the growth of the bacterium. The biomass degradation enzyme may be selected from amylases, xylanases, cellulases, laccases, arabinofuranosidases, alpha-glucuronidases, and esterases. Preferred examples of esterases include, without limitation, ferulic acid esterases or acetyl xylan esterases. A specific example of arabinofuranosidase includes, without limitation, alpha-L-arabinofuranosidase. In a preferred embodiment, the biomass degradation enzymes are selected from amylases, cellulases and arabinofuranosidases.

In a preferred embodiment, the unit comprises three genes under the control of the single promoter. Specific examples of such units of the invention contain:
2 distinct amylase genes, or
1 amylase gene and 1 arabinofuranosidase gene, or
2 distinct amylase genes and 1 arabinofuranosidase gene.

Amylases are involved in the hydrolysis of polysaccharides, particularly starch. Starch is a carbohydrate consisting of a large number of glucose units joined together by 1-4 and 1-6 glycosidic bonds. The term “amylases” includes polypeptides having alpha-amylase, beta-amylase, glucoamylase, alpha-glucosidase or pullulanase (glycosyl hydrolase) activities. Alpha-amylases have the ability to hydrolyze internal alpha-1,4-glucosidic linkages in starch to produce smaller molecular weight malto-dextrins. Glucoamylases have ability to hydrolyse glucose polymers linked by a-1,4- and a-1,6-glucosidic bonds. Glucoamylases have the ability to release beta-D-glucose from glucans.

The amylase gene for use in the invention is preferably a gene encoding an alpha amylase. More preferably, the amylase gene encodes an amylase derived from a Deinococcus bacterium. In this regard, applicant has identified novel amylase genes from Deinococcus bacteria, with improved properties, the sequences of which are represented in SEQ ID NOs: 2 and 4 (amino acid sequences).

In a preferred embodiment, the transcriptional unit of the invention comprises a gene encoding an alpha amylase comprising the amino acid sequence of SEQ ID NO: 2 or 4, or a fragment or variant thereof.

Functional analyses conducted by the inventors have shown that the alpha amylase of SEQ ID NOs 2 and 4 exhibit different substrate specificity, so that by combining these two amylases in a bacterium of the invention, the spectrum of activity is increased.

Cellulases are enzymes that catalyze the hydrolysis of cellulose or hemicellulose, a major component of hardwood and softwood. Cellulases may be of different types, such as endoglucanases, endocellulases, cellobiohydrolases (CBH) or cellobiosidases, or (3-Glucosidases (cellobiases; BGL). The cellulase gene for use in the present invention is preferably a gene encoding an endocellulase or an exocellulase. More preferably, the cellulase gene encodes a cellulase derived from a Deinococcus bacterium. In this regard, the applicant has identified novel cellulase genes from Deinococcus bacteria, with improved properties, the sequences of which are represented in SEQ ID NOs: 8 and 10 (amino acid sequences).

In a preferred embodiment, the transcriptional unit of the invention comprises a gene encoding a cellulase comprising the amino acid sequence of SEQ ID NO: 8 or 10, or a fragment or variant thereof.

The arabinofuranosidase gene may be any gene encoding an arabinofuranosidase, more preferably an arabinofuranosidase derived from a Deinococcus bacterium. In this regard, the applicant has identified a novel arabinofuranosidase gene from Deinococcus bacteria, with improved properties, the sequence of which is represented in SEQ ID NO: (nucleic acid) and 6 (amino acid).

In a preferred embodiment, the transcriptional unit of the invention comprises a gene encoding an arabinofuranosidase comprising the amino acid sequence of SEQ ID NO: 6, or a fragment or variant thereof.

Examples of biomass degradation transcriptional units of the invention comprise, under the control of a single promoter, at least two genes selected from:
a gene encoding an alpha amylase comprising the amino acid sequence of SEQ ID NO: 2 or 4, or a fragment or variant thereof;
a gene encoding an arabinofuranosidase comprising the amino acid sequence of SEQ ID NO: 6 or a fragment or variant thereof and
a gene encoding a cellulase comprising the amino acid sequence of SEQ ID NO: 8 or 10, or a fragment or variant thereof.

More specific examples of biomass degradation transcriptional units of the invention are described in the examples, and comprise, under the control of a single promoter, and in the 5′ to 3′ order:
a gene encoding an alpha amylase comprising the amino acid sequence of SEQ ID NO: 2, or a fragment or variant thereof, and a gene encoding an alpha amylase comprising the amino acid sequence of SEQ ID NO: 4 or a fragment or variant thereof or
a gene encoding an alpha amylase comprising the amino acid sequence of SEQ ID NO: 2, or a fragment or variant thereof, and a gene encoding an arabinufuranosidase comprising the amino acid sequence of SEQ ID NO: 6 or a fragment or variant thereof or
a gene encoding an alpha amylase comprising the amino acid sequence of SEQ ID NO: 2, or a fragment or variant thereof, a gene encoding an alpha amylase comprising the amino acid sequence of SEQ ID NO: 4 or a fragment or variant thereof, and a gene encoding an arabinufuranosidase comprising the amino acid sequence of SEQ ID NO: 6 or a fragment or variant thereof.

The invention shows the above gene configurations allow proper expression of integrated genes for optimal ethanol production.

The unit may comprise alternative or further genes, encoding for instance laccases, xylanases or esterases. Specific examples of such other genes, isolated by applicant from Deinococcus or related bacteria, are disclosed in PCT/EP2011/069669 and PCT/EP2011/069670, incorporated by reference.

The expression of the genes in the transcriptional unit is regulated by a single promoter. The promoter may be homologous to the host (e.g, a promoter from a Deinoccocus gene) or heterologous (e.g., from a distinct origin, such as a distinct bacterium, a phage, a synthetic or hybrid promoter, etc.). Preferred promoters are homologous. In this regard, various promoters have been studied and used for gene expression. Examples of suitable Deinococcus promoters include PtufA and PtufB promoters from the translation elongation factors Tu genes tufA (DR0309) and tufB (DR2050), the promoter of the resU gene located in pI3, and the promoter region PgroESL of the groESL operon (Lecointe et al, 2004; Meima et al, 2001), or derivatives of such promoters.

The invention show that suitable levels of expression of the genes in the transcriptional unit are obtained when the genes are placed under the control of a promoter selected or derived from PtufA, PtufB or PgroESL.

The transcription unit may further comprise additional regulatory sequences, such as for instance terminators and/or enhancers.

The transcriptional unit may, in addition, comprise further genes under the control of distinct promoters. In this regard, it is possible to insert in the transcriptional unit additional genes encoding biomass degradation enzymes, regulated by the same or distinct promoter(s).

In a particular and preferred embodiment, the transcriptional unit of the invention further comprises at least one additional biomass degradation gene under the control of a second promoter. In a further preferred embodiment, the transcriptional unit comprises 2 additional biomass degradation genes under the control of a promoter. The invention shows that such a reconstructed biomass degradation unit can be assembled in one location in the genome of a Deinococcus strain. The invention shows the configuration in operons provide optimal gene expression levels to ethanol production. The invention shows such bacteria are viable and stable. These bacteria therefore represent very potent biocatalysts for biofuel production.

In a particular embodiment, the invention relates to a Deinococcus or related bacterium comprising a reconstructed biomass degradation transcription unit, wherein said unit comprises:
(i) under the control of a single promoter, 2 amylase genes, or 1 amylase gene and 1 arabinofuranosidase gene, or 2 amylase genes and 1 arabinofuranosidase gene; and
(ii) under the control of a second promoter, at least one cellulase gene, preferably 1 endocellulase gene and 1 exocellulase gene.

These particular combinations and arrangement of genes allows improved expression and activity in the bacterium.

Transcriptional units may be either prepared in vitro and then inserted into the cell, or constructed in the cell by sequential insertion of the genes. Conventional recombinant DNA techniques can be used for DNA manipulation, cloning, and cell transformation. In particular, the nucleic acid(s) may be inserted into the genome of the bacterium, or inserted as (autonomously) replicating molecules, e.g., on a plasmid, episome, artificial chromosome, etc.

In a preferred embodiment, the transcriptional unit is integrated into the genome of the bacterium. For that purpose, the construct is cloned into one or several integrative cassettes suitable for integration into the genome of a Deinococcus bacterium. Such an integrative cassette comprises, typically, the nucleic acid linked to (or flanked by) one or several sequences allowing integration, preferably site-specific integration. Such sequences may be for instance nucleic acid sequences homologous to a targeted region of the genome, allowing integration through crossing over.

Insertion may be targeted to non essential (e.g., non coding regions) of the genome. However, in a preferred embodiment, insertion is targeted to specific genes within the genome of the bacterium. In this regard, a particular bacterium of the invention comprises a reconstructed biomass degradation transcriptional unit integrated into its genome, in replacement of all or part of an endogenous gene encoding an amylase. The invention shows replacement of the endogenous gene by a transcription unit of the invention further improves the properties of the bacterium. In this context, the term “part of the gene” means any portion of the gene the deletion of which being sufficient to cause inactivation of the gene in the cell.

Various techniques can be used to insert a nucleic acid into Deinococcus or related bacteria, as disclosed for instance in WO2010/130806. In particular, they may be inserted through natural transformation (which can be further enhanced in presence of calcium chloride) or electroporation.

Alternative cloning sites include, preferably, in replacement of a gene selected from a phosphate acetyl transferase gene, an Alanine dehydrogenase gene, a glucose dehydrogenase gene, a phosphoenolpyruvate carboxykinase gene, a phosphoenolpyruvate carboxylase gene, or a malate dehydrogenase gene.

In an alternative embodiment, although less preferred, the transcription unit may be cloned into a suitable vector, which may be replicative in Deinococcus. Typical vecors contain, in addition to the cloned insert, a selection gene (e.g., antibiotic resistance, a dye, etc.) and an origin of replication effective in Deinococcus. Examples of such vectors include pMD66, pI3, pRAD1 and pUE30. pMD66 is a large vector (27 kb) for D. radiodurans and E. coli containing a 12 kb fragment of pI3 (Daly et al, 1994). pI3 was described by Masters and Minton (1992). pRAD1 is a D. radiodurans-E. coli shuttle plasmid containing a minimal replicon for D. radiodurans (Meima and Lidstrom, 2000). pUE30 is an endogenous plasmid derived from a strain of D. radiopugnans which is able to replicate in Deinococcus (see US2003/0175977).

The invention also relates to a method for producing a Deinococcus or related bacterium, or an ancestor thereof, the method comprising:

a) providing a parent Deinococcus or related bacterium;
b) simultaneously or sequentially inserting into the genome of said parent bacterium at least 2 genes under the control of a single promoter, said at least 2 genes encoding distinct biomass degradation enzymes, and
c) selecting a bacterium of b) which expresses both said at least 2 genes.

Bacteria having inserted the nucleic acids may be selected according to techniques known per se. Expression of the genes may be verified using (e.g. quantitative) PCR and production of these enzymes may be verified by Western blot or by enzymatic assays known per se in the art and illustrated in the examples.

As disclosed in the experimental section, several Deinococcus bacteria containing a reconstructed biomass degradation transcriptional unit have been produced. These bacteria can be cultivated, are viable and stably contain the inserted unit. Stability is preferably such that more than 95% of the bacteria still contain the unit after 2 growth cycles. A further advantage of the invention is that the biomass degradation transcription unit can be made entirely of Deinococcus derived genetic material. As a result, the bacterium is more stable, more effective, not a GMO, and more adapted to extreme culture conditions. Also, the particular enzymes identified and characterized by the inventors exhibit potent and complementary activities which confer on the bacteria remarkable activities.

### Alcohol Production Transcriptional Unit

In a preferred embodiment, the bacteria of the invention comprise, in addition to the biomass degradation transcriptional unit, a recombinant alcohol production transcriptional unit. Such bacteria therefore express optimized gene combinations to produce biofuels or metabolites from biomass.

The recombinant alcohol production transcriptional unit comprises preferably at least one gene encoding an Alcohol dehydrogenase (adh) and/or a Pyruvate decarboxylase (pdc).

Pyruvate decarboxylases (PDC, EC: 4.1.1.1) catalyse the mono-oxidative decarboxylation of pyruvate to acetaldehyde and carbon dioxide. Alcohol dehydrogenases (ADH, EC: 1.1.1.1) catalyse the conversion of acetaldehyde to ethanol. The insertion of and ADH and/or PDC gene into a Deinococcus has been reported in a prior application filed by applicant (WO2010/130806). In order to create or improve this metabolic pathway, genes encoding a PDC and/or an ADH have now been cloned and successfully introduced into Deinococcus or related bacteria of this invention, having a reconstructed biomass degradation unit.

More particularly, a gene encoding a functional PDC has been prepared. Such a nucleic acid molecule can comprise all or a portion of the sequence of a natural or synthetic or mutant PDC gene, as long as the nucleic acid molecule encodes a protein that catalyses the mono-oxidative decarboxylation of pyruvate to acetaldehyde and carbon dioxide. PDC is present in plants, fungi and yeast but is rare in bacteria. No apparent PDC has been found in D. radiodurans genome that was fully sequenced. PDC genes have been identified in various strains, such as in Zymomonas mobilis (Brau and Sahm, 1986; Conway et al, 1987a; Neale et al, 1987), in Acetobacter pasteurianus (Genbank: AF368435) (Chandra et al, 2001), in Sarcina ventriculi (Genbank: AF354297) (Lowe and Zeikus, 1992) and in Zymobacter palmae (Genbank: AF474145) (Raj et al, 2002).

In a preferred embodiment, the PDC nucleic acid comprises the sequence of all or part of a bacterial PDC gene. In a specific embodiment, the nucleic acid comprises the sequence of a PDC gene from Zymomonas mobilis (ZmPDC, ZMO1360). ZmPDC gene sequence comprises 1707 base pairs and is represented in SEQ ID NO: 15. A modified gene sequence, optimized for expression in Deinococcus, has been synthesized by the inventors, which is presented in SEQ ID NO: 16.

ADH genes have been cloned from different organisms including, without limitation, Zymomonas mobilis (Ingram et al, 1987), Lactobacillus brevis (Liu et al, 2007), or Geobacillus stearothermophilus (Genbank: Z25544) (Talarico et al, 2005). ADH genes from Deinococcus have also been isolated and cloned by the inventors. The sequence of these genes and encoded ADH enzymes are disclosed in SEQ ID NOs: 11-14.

In order to create a bacterium encoding an ADH activity, a gene encoding a functional ADH is prepared, which can comprise all or a portion of the sequence of a natural or synthetic or mutant ADH gene, as long as the nucleic acid molecule encodes a protein that catalyses the conversion of acetaldehyde to ethanol.

In a preferred embodiment, the ADH nucleic acid comprises the sequence of all or part of a bacterial ADH gene. In a specific embodiment, the nucleic acid comprises the sequence of an ADH gene from Zymomonas mobilis (ZmADH, ZMO1596). ZmADH II comprises 1152 base pairs and the sequence thereof is depicted in WO2010/130806 (see also SEQ ID NO: 17). A modified gene sequence, optimized for expression in Deinococcus, has been synthesized by the inventors, which is presented in SEQ ID NO: 18.

In another preferred embodiment, the nucleic acid encodes an ADH comprising all or part of amino acid sequence SEQ ID NO: 12 or 14.

According to another specific embodiment, the alcohol production transcriptional unit comprises at least one gene encoding an NADP-dependent ADH or PDC.

A metabolic flux ratio analysis showed that some Deinococcus exhibit the surprising property of consuming glucose mainly through the Pentose Phosphate Pathway, indicating that an important part of NADPH is produced as redox potential. In order to use this NADPH pool, a NADP-dependent ADH gene may be used. In this regard, the ethanol unit may comprise ADH gene HUC 22-1 derived from Moorella sp (e.g., comprising all or a functional part of SEQ ID NO: 19) or Tzadh from Zymomonas (comprising all or a functional part of SEQ ID NO: 20). The invention shows expression of such genes in Deinococcus bacteria is particularly useful since these bacteria have a high level of NADPH flux.

An object of the invention therefore also resides in a Deinococcus or related bacterium, wherein said bacterium comprises at least one recombinant nucleic acid sequence encoding an NADP-dependent ADH or PDC.

A further object of the invention is a Deinococcus or related bacterium, wherein said bacterium comprises at least one recombinant nucleic acid sequence encoding an NADP-dependent ADH and at least one recombinant nucleic acid sequence encoding an NAD-dependent ADH.

The nucleic acid(s) may be inserted into the genome of the bacterium, or inserted as (autonomously) replicating molecules, e.g., on a plasmid, episome, artificial chromosome, etc., as disclosed above. In a preferred embodiment, the ethanol production transcriptional unit is inserted in the genome of said bacterium in replacement of an endogenous gene, more preferably in replacement of a phosphate acetyl transferase gene.

In this regard, an object of the invention resides in a Deinococcus or related bacterium, wherein said bacterium contains an inactivated phosphate acetyl transferase gene (pt a). AcetylCoA, which is produced during glycolysis, can be used for ethanol production as well as for acetate formation which is a secondary competitive pathway. By deleting pta gene, the acetate production should be reduced (or abolished) to favor ethanol production. The invention shows this gene may be deleted without altering the growth of the bacteria, while improving the ability of the bacteria to produce biofuels.

Expression of appropriate PDC or ADH may be verified using quantitative PCR and production of these enzymes may be verified by Western blot or by enzymatic assays known per se in the art. PDC activity can be measured by analyzing the reduction of NAD⁺ and ADH activity can be measured by analyzing the reduction of NAD⁺ or oxidation of NADH due to the activity of these enzymes (Conway et al, 1987a and b).

### Deletion of Competing Pathways

The properties of the bacteria of the invention can be further improved by deleting or altering competing reactions or pathways in the cell.

The inventors have now created novel bacteria in which genes involved in competing pathways have been inactivated. Such bacteria exhibit further improved efficacy in the production of biofuels from biomass, by using more of their energy into the desired pathways. More specifically, particular objects of this invention resides in Deinococcus or related bacteria comprising an inactivated endogenous gene selected from a phosphate acetyl transferase gene, an Alanine dehydrogenase gene, a glucose dehydrogenase gene, a phosphoenolpyruvate carboxykinase gene, a phosphoenolpyruvate carboxylase gene, and/or a malate dehydrogenase gene.

In a particular embodiment, the target gene is deleted, in all or in part, and does not encode a functional protein. The target gene may be inactivated in said bacterium or an ancestor thereof, by homologous recombination, gene replacement, or targeted mutagenesis, or any other technique known per se in the art.

In a preferred embodiment, the gene is inactivated by deletion of at least part of said gene, which may be replaced by heterologous nucleic acid (e.g., a selection marker).

In a preferred embodiment, the bacterium of the present invention lacks a portion of said gene, preferably at least 100 consecutive nucleotides thereof, more preferably at least 200, 300, 400 or 500. In the examples, a defective Deinococcus strain has been produced, which lacks the entire phosphate acetyl transferase gene. This strain has been prepared by double crossing-over using a particular construct comprising an ethanol-production transcription unit flanked by two regions homologous to portions of the gene. Typical homologous regions should be long enough to allow hybridization and crossing-over, e.g., above 200 nucleotides, preferably above 300 nucleotides, typically between 300 and 700. Such constructs represent particular object of the present invention.

In this regard, the invention also relates to a method for producing a Deinococcus bacterium as defined above, or an ancestor thereof, the method comprising:
providing a (parent) Deinococcus bacterium;
Treating the bacterium to inactivate an endogenous gene selected from a phosphate acetyl transferase gene, an Alanine dehydrogenase gene, a glucose dehydrogenase gene, a phosphoenolpyruvate carboxykinase gene, a phosphoenolpyruvate carboxylase gene, and/or a malate dehydrogenase gene, and
Selecting a bacterium having said gene inactivated.

### Culture, Compositions and Uses

Bacteria of the invention can be prepared from any species of Deinococcus or related bacteria. Examples are listed supra in the present application. They are preferably Deinococcus bacteria of a species selected from D. geothermalis, D. cellulolysiticus, D. deserti, D. murrayi, and D. radiodurans.

Illustrative examples of parent strains suitable for use in the invention include, without limitation, e.g. D. geothermalis DSM 11300 (DRH05), D. geothermalis DSM 11301 (DRH06), D. geothermalis DSM 11302 (DRH07), D. geothermalis HAMBI 2481 (DRH37), D. geothermalis HAMBI 2480 (DRH38), D. geothermalis HAMBI 2411 (DRH39), D. geothermalis HAMBI 2791 (DRH41), D. geothermalis M36-7D; D. radiodurans R1 (ATCC 13939); D. murrayi M11-9D (CNCM 1-4155); or D. murrayi M13-1A (CNCM 1-4157).

Other strains of Deinococcus are deposited in public collections, or may be obtained by the skilled artisan, which can be used to implement the invention.

The bacteria of the present invention may be cultivated and/or maintained in any suitable culture medium and/or device. Examples of such medium include complex glucose medium or defined medium as disclosed in the examples, such as e.g., defined medium sucrose, defined medium starch. Suitable medium are also commercially available.

A further object of the invention relates to a composition comprising a bacterium as defined above and at least one other bacterium.

A further object of the invention relates to a composition comprising a bacterium as defined above and a culture medium.

A further object of the invention relates to an enzymatic extract of a bacterium as defined above. The enzymatic extract contains at least a biomass degradation enzyme encoded by the reconstructed unit.

The invention also concerns a biotacalyst comprising a bacterium or an extract thereof as defined above.

The invention further resides in a process for transforming biomass, comprising exposing a biomass to a bacterium, or an extract, or a composition as defined above.

A further object of the invention is a process for producing a biofuel, comprising exposing a sugar or biomass to a bacterium, or an extract, or a composition as defined above. Preferably, the process comprises a step of collecting biofuel produced.

The invention also relates to the use of a bacterium as defined above for producing a biofuel or metabolite.

The substrate may be any culture medium or various types of biomass or products derived therefrom. In particular, the biofuel may be produced from renewable resources, especially plant or animal biomass, or from municipal and industrial wastes.

More preferably, the method of the invention is used for the production of ethanol.

The method of the invention may be performed in a reactor of conversion. By “reactor” is meant a conventional fermentation tank or any apparatus or system for biomass conversion specially designed to implement the invention and therefore consisting in particular of bioreactors, biofilters, rotary biological contactors, and other gaseous and/or liquid phase bioreactors, especially those adapted for the treatment of biomass or biomass derivatives. The apparatus which can be used according to the invention can be used continuously or in batch loads.

In the reactor, to implement the method of the invention, at least one bacterium of the invention, or bacterial extract thereof, is used, whilst said reactor is arranged and supplied so that physicochemical conditions are set up and maintained therein so that said bacterium is operational for the application under consideration and so that, optionally, bacterial growth is possible and preferably promoted therein.

The process may be conducted under aerobiosis, anaerobiosis or under microaerobiosis, depending on the substrate and bacterium. An advantage of the invention relates in the ability of the bacteria of the invention to resist stressful conditions, including the presence of ethanol in the culture medium. The process of the invention may thus preferably be performed at a temperature of about 40° C. or more, particularly a temperature comprised between 40-70° C.; under acid pH conditions, and/or in the presence of ethanol.

Further aspects and advantages of the invention will be disclosed in the following examples, which should be considered as illustrative and do not limit the scope of this application.

### EXAMPLES

### A. Materials and Methods

### Bacterial Strains and Growth Conditions:

Escherichia coli (E. coli) strains SCS110, JM109 or DH5a were used to propagate plasmids. They were cultivated at 37° C. and 200 RPM in Luria-Bertani (LB) Broth (per liter: Tryptone 10 g, Yeast extract 5 g, Sodium chloride 10 g). Solid media was prepared by addition of Agar 1.5%.

Deinococcus bacteria were cultivated at 45° C. and 200 RPM in PGY. The composition of the PGY medium is the following, per liter: Peptone (10 g), Yeast extract (5 g) and Glucose (1 g). Composition of the solid media is, per liter: Peptone (10 g), Yeast extract (5 g), Glucose (1 g) and Agar (15 g).

When needed, LB or PGY media were supplemented with appropriate antibiotics:
chloramphenicol, at a final concentration of 3 μg/ml for D. geothermalis, and 30 μg/ml for E. coli
Bleocin, at a final concentration of 6 μg/ml for D. geothermalis, and 10 μg/ml for E. coli
Ampicillin, at a final concentration of 100 μg/ml for E. coli

### Transformation:

E. coli transformation was done using commercial competent cells SCS110 from Stratagene or JM109 from Promega.

For Deinococcus cells, a fresh culture in stationary phase was diluted 100 times in 50 ml of PGY. Cells were grown until late exponential phase (OD₆₀₀ₙₘ=0.8); the pellet was resuspended in an appropriate volume of ice cold 2×PGY/10% v/v Glycerol/30 mM CaCl₂. For transformation, desired amount of plasmid DNA was added to 100 μl of the cells. The mixture was incubated 30 minutes on ice transferred at 42° C. for 90 seconds and back to ice for 5 minutes. 200 μl of fresh 2×PGY medium was added and the transformants were shaked at 200 RPM and 37° C. during 2 hours. They were serially diluted and spread on appropriate selective PGY plates.

### DNA Manipulation:

Plasmid minipreparation from E. coli cells was done using the QIAGEN minipreps DNA purification system and midipreparation was done using the Plasmid DNA purification NucleoBond® Xtra Midi Plus EF kit from Macherey-Nagel. These preparations were done from 3-100 ml of E. coli culture in stationary phase.

Genomic DNA extraction from Deinococcus was done using the DNeasy® Blood and Tissue commercial kit from Qiagen. These preparations were done from 5 ml of stationary phase cultures.

The oligonucleotides were synthesized by Eurogentec. The polymerases used for PCR amplification were the PHUSION Hi-Fidelity polymerase from Finzyme; and the KOD Xtreme-hot start DNA polymerase from Novagen for overlapping PCRs. PCR fragments were cleaned up using the Wizard SV Gel and PCR Clean-Up System kit from Promega.

Genetic material were separated by agarose gel electrophoresis. DNA was quantified with a Biophotometer from Eppendorf.

DNA inserts were synthetized by Genecust Europe and cloned into appropriate vector.

Method of Genetic Insertion into Deinococcus Chromosome:

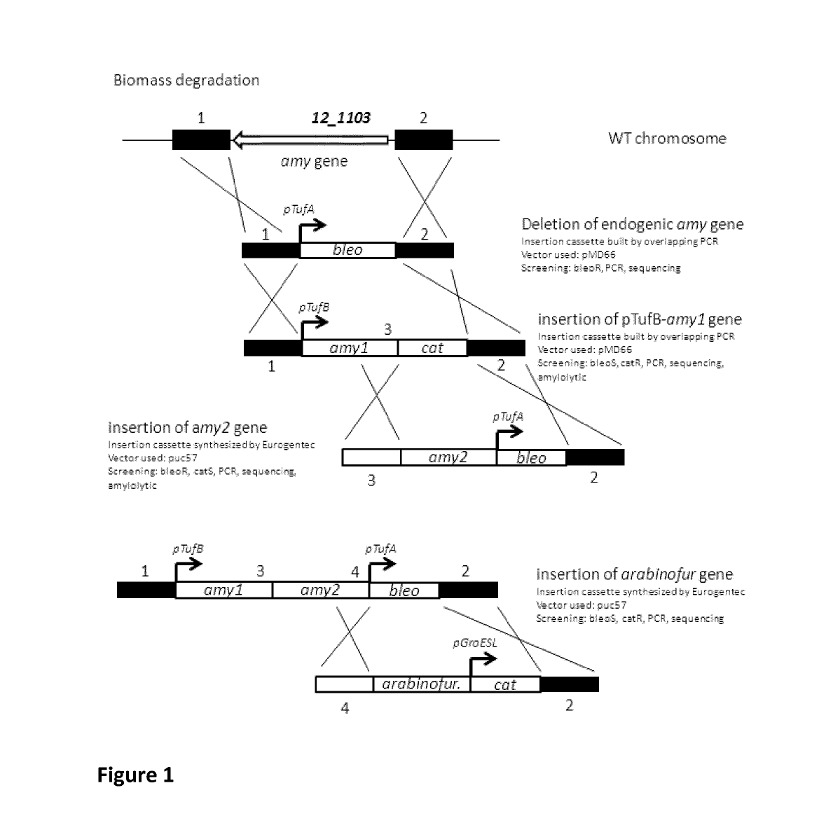

Insertion of DNA fragments into the chromosome of Deinococcus was performed using homologous recombination mechanism. Cassettes of insertion were designed as follows: the DNA sequence of the region that had to be inserted was flanked by 500 bp regions homologous to the sequences upstream or downstream the chromosomic target (see FIG. 1).

For the first 2 steps (deletion of the endogenic α-amylase encoding gene 12_{—}1103 and insertion of pTufB-amyl), insertion cassettes were carried by pMD66 thermosensitive shuttle vector, transformed into Deinococcus and high temperature exposure (52° C. for 4 days) was used to allow for chromosomic insertion and plasmid loss.

For all other insertions/deletions insertion cassettes were cloned into puc57 suicide vector and transformed into Deinococcus. Transformants that had incorporated the region of interest into the chromosome were selected on PGY medium containing the appropriate antibiotic.

Correct insertions/deletions were checked by PCR on genomic DNA, marker replacement (Chloramphenicol to bleocin or bleocin to chloramphenicol when possible), and sequencing of the modified chromosome on the region of interest.

FIG. 1 is a schematic representation of the first insertion steps and methods used to insert biomass degradation genes and check for correct insertions.

### Alcohol Dehydrogenase Activity Test:

4 ml of pararosaniline (Sigma) at 2.5 mg/ml in absolute ethanol were added to 200 ml of LB agar containing 50 mg of sodium bisulfate (Conway et al, 1987b). 2-days-old D. radiodurans cells grown on TGY agar plates (supplemented if necessary with the appropriate antibiotic) were plated on the indicator plates and incubated at 37° C. for 2 to 3 hours.

### Metabolites Production:

This method enables the evaluation of the ability of genetically modified microorganisms to produce metabolites of interest from biomass or a derivative of biomass.

The test is carried out at 30° C.

From pre-cultures (in stationary phase) prepared in Complex medium Glucose, 6 ml of enriched medium are seeded (seeding at 1% v/v).

The enriched culture mediums tested are Complex Medium Glucose, Defined Medium Sucrose, Defined Medium Starch.

Complex Medium Glucose contains: peptone 2 g/L, yeast extract 5 g/L and glucose 10 g/L in osmosed water: solution sterilized by autoclaving (15 minutes at 120° C.). To this solution are added the following solutions: MOPS buffer solution (10×) pH7 [acid MOPS 400 mM, NH₄Cl 200 mM, NaOH 100 mM, KOH 100 mM, CaCl₂ 5 μM, Na₂SO₄ 2.76 mM, MgCl₂ 5.28 mM]; micronutrients (10000×) [(NH₄)₆(Mo₇)24 300 mM, H₃BO₃ 4 mM, CoCl₂ 0.3 mM, CuSO₄ 0.1 mM, MnCl₂ 2.5 mM, ZnSO₄ 0.1 mM]; FeCl₃(100×) 2 mM in C₆H₅Na₃O₇ 20 mM; K₂HPO₄ 1 g/L: solutions sterilized by filtration (0.2 μm).

Defined Medium contains: carbon source 10 g/L in osmosed water: solution sterilized by autoclaving (15 minutes at 120° C.). To this solution are added the following solutions: MOPS buffer solution (10×) pH7 [acid MOPS 400 mM, NH₄Cl 200 mM, NaOH 100 mM, KOH 100 mM, CaCl₂ 5 μM, Na₂SO₄ 2.76 mM, MgCl₂ 5.28 mM]; micronutrients (10000×) [(NH₄)₆(Mo₇)24 300 mM, H₃BO₃ 4 mM, CoCl₂ 0.3 mM, CuSO₄ 0.1 mM, MnCl₂ 2.5 mM, ZnSO₄ 0.1 mM]; FeCl₃(100×) 2 mM in C₆H₅Na₃O₇ 20 mM; K₂HPO₄ 1 g/L: solutions sterilized by filtration (0.2 μm).

To these culture mediums, except for wild type strains, chloramphenicol is added before the seeding: 3 μg/mL the culture medium.

Cultures are performed both in aerobiosis and anaerobiosis (Biomerieux, Genbag).

Cultures in aerobiosis condition are left in an incubator, at 30° C., under agitation, for 7 days. The cultures are then centrifuged for 10 minutes at 4000 rpm. Supernatants are filtered (0.2 μm), poured into other tubes, and placed at −80° C.

Cultures in anaerobiosis condition are left in an incubator, at 30° C., for 4 weeks. The cultures are then centrifuged for 10 minutes at 4000 rpm. Supernatants are filtered (0.2 μm), poured into other tubes, and placed at −80° C.

Gas Chromatography FID analysis (Varian CP-WAX 57 CB 25 m*0.32 mm column) was used to quantify alcohols. Organic acids were quantified by Liquid Chromatography Mass Spectroscopy (MicrOTOF-QII Bruker) or Capillary Electrophoresis (5 mM 2,6-pyridinedicarboxylic acid 0.5 mM Cetyltrimethylammonium bromide; 5.6 pH adjusted buffers/61 cm length, 50 μm diameter capillary Agilent). Residual glucose was quantified by HPLC coupled with refractometry (Phenomenex LUNA 3 μm NH₂ 100A 150*4.6 mm column, acetonitrile/H₂O 85:15 mobile phase).

Acids and ethanol productions were monitored for the different strains after 7 days of growth in whole wheat 3%- or 6%-containing medium, in aerobic conditions.

### B. Engineering of a Deinococcus Strain with a 2-Genes Reconstructed Biomass Degradation Transcriptional Unit

D. geothermalis DSM 11300 is used as parent strain. In separate sets of experiments, the same protocol is applied to other Deinococcus or related bacteria as listed below:

DRH05 D. geothermalis DSM 11300
DRH06 D. geothermalis DSM 11301
DRH07 D. geothermalis DSM 11302
DRH37 D. geothermalis HAMBI 2481
DRH38 D. geothermalis HAMBI 2480
DRH39 D. geothermalis HAMBI 2411
DRH41 D. geothermalis HAMBI 2791
M36-7D D. geothermalis
DR1 D. radiodurans ATCC 13939
M11-9D D. murrayi CNCM 1-4155
M13-1A D. murrayi CNCM 1-4157

### B1. Deletion of the Wt α-Amylase 12_{—}1103 (Only) Encoding Gene

A DNA fragment containing the Bleocin resistance gene (bleo) placed under the control of a pTufA promoter was inserted into the chromosome of the parent strain, replacing the endogenic α-amylase encoding gene 12_{—}1103 by homologous recombination, giving strain DG_{—}06. Insertion was carried out using 500 bp fragments homologous to regions directly upstream and downstream the endogenic α-amylase encoding gene. Complete replacement of the entire α-amylase Open Reading Frame from ATG to stop codon was confirmed by PCR and sequencing.

B2. Insertion of the α-Amylase Encoding Gene M23r-3A.18_{—}109 (Amyl), from Strain M23-3A, at the Amy Locus

A DNA fragment harboring the α-Amylase encoding gene M23r-3A.18_{—}109 (amyl—SEQ ID NO: 1) in operon with a chloramphenicol resistance gene (cat, SEQ ID NO: 23) and placed under the pTufB promoter was inserted into the chromosome of strain DG_{—}06 (amy::ptufA-bleo), replacing pTufA-bleo and giving strain DG_{—}02 (amy::pTufB-amyl-cat). Insertion was carried out using 500 bp fragments homologous to regions directly upstream and downstream the pTufA-bleo cassette, therefore replacing the entire pTufA-bleo cassette. Insertion was checked by marker replacement (bleo to cat) and PCR. The amyl encoding sequence is fused to a N-terminal signal peptide sequence to allow for secretion of the protein.

B3. Insertion of the α-Amylase Encoding Gene M23r-3A.305_{—}673.Amy2), from Strain M23-3A, Downstream the Amyl Gene

A DNA fragment containing the α-Amylase encoding gene M23r-3A.305_{—}673. amy2, SEQ ID NO: 3) and the pTufA-bleo resistance cassette, was inserted into the chromosome of strain DG_{—}02 (amy::pTufB-amyl-cat), directly downstream the amyl gene and replacing the cat resistance gene. Insertion was carried out using 500 bp fragments homologous to regions directly upstream and downstream the cat gene, therefore deleting the entire cat gene. The resulting strain, DG_{—}04 (amy::pTufB-amyl-amy2-pTufA-bleo), harbors an operon of both amylases placed under the control of pTufB promoter. Insertion was checked by marker replacement (cat to bleo) and the operon structure confirmed by PCR.

### B4. Amylases Expression and Amylolytic Activity in Reconstructed Bacteria

a—Amylolytic Assay on Plates

Cells were grown in rich PGY medium to late exponential phase. 2 to 8 μl of culture (quantity normalized according to OD600 nm) was then spotted onto minimal medium plates containing 0.5% starch and incubated at 37° C.

After 3 days, amylolytic halos were visualized using Gram's iodine coloration

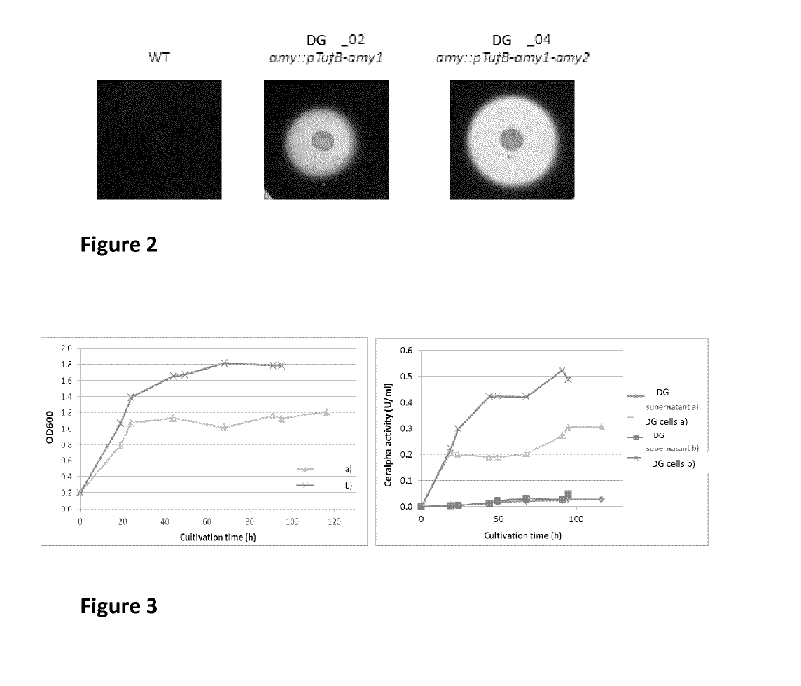

The results are presented FIG. 2 and show the presence of a strong amylase activity in the reconstructed strains.

b—Characterization of Amylolytic Strain DG_{—}04

Strain DG_{—}04 (amy::pTufB-M23r2A.18_{—}109-M23r-2A.305_{—}673-pTufA-bleo) containing α-amylases M23r-2A.18_{—}109 and M23r-2A.305_{—}673 was grown on defined minimal medium containing 0.5% soluble starch (Sigma S976). The medium was enriched with 5 mM CaCl₂ and 6 μg/ml Bleomycin (Calbiochem 203408). Cultivations were carried out in 5 L shake flasks containing 2 L of medium. Samples were taken daily and measured for OD600 and α-amylase activity.

As presented in FIG. 3, an alpha amylase activity is observed, and most of the activity was detected from the surface of the cells.

Triton X-100 extraction was tested for the cell pellet to release the α-amylase activity located on the cell surface. Cells were suspended in 100 mM MOPS buffer containing 2 mM CaCl₂ and Triton X-100 was added to a final concentration of 0.1%. Samples were mixed well and incubated at room temperature for 15 min. After incubation, cells were removed by centrifugation and the supernatant was analyzed for α-amylase activity. Approximately 45% of the α-amylase activity could be released from the cell surface by Triton extraction.

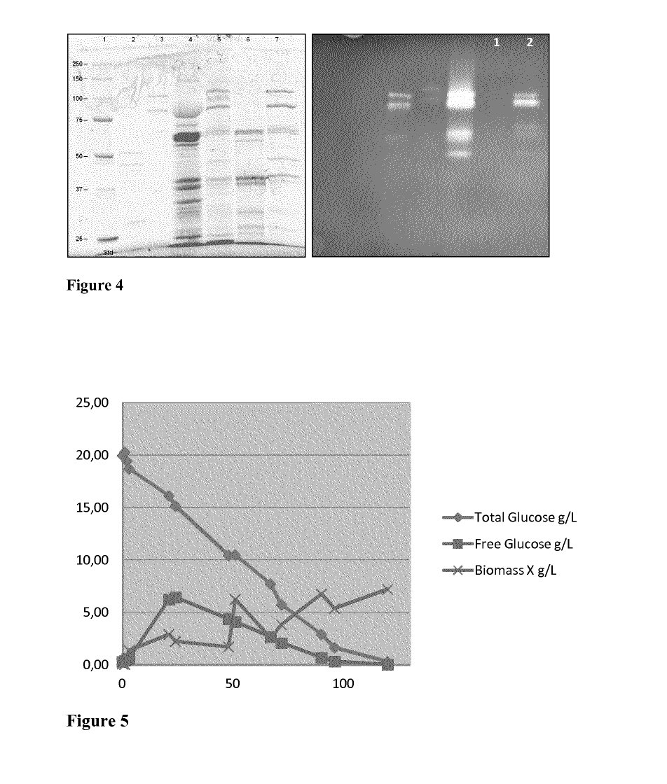

Concentrated DG_{—}04 culture supernatants, cells and Triton extracts were further analyzed by SDS-PAGE and zymogram technique (FIG. 4). Amylolytic Deinococcus strain M23-3A was used as a reference strain. DG_{—}4 showed five active bands on the zymogram gel. Two major bands were detected near 100 kDa whereas two other active bands situated near 65 kDa. The theoretical molecular weight of the target α-amylase (2^{nd} α-amylase) is 109 kDa and one of the major bands most probably represents the target enzyme. One weak band was observed near 50 kDa which corresponds with the molecular weight of the 1^{st} α-amylase (MW=53 kDa). The parent strain contains several amylolytic genes. However, clear signal sequences have been identified only from the two inserted α-amylase genes. Thus, the unidentified bands may originate from proteolytic degradation of the extracellular enzymes or partial cell lysis releasing intracellular activities. Gel artifacts and protein aggregates are also possible explanation for the zymogram results due to the non-reducing conditions used in the analysis. Highest activities with the parent strain were detected in the cell pellet sample (lane 5) and as the activity can partly be released from the cell surfaces with Triton extraction it is suggested that the purification of the 2^{nd} α-amylase will be performed from the Triton extracts. With the amylolytic wild strain M23-3A very weak activity with a molecular weight of 50 kDa was detected in the culture supernatant (lane 2) whereas two active bands near 100 kDa were observed in the cell pellet sample (lane 4). The results suggest that, in wild strain M23-3A, the smaller M23r-2A.18_{—}109 α-amylase (1^{st} α-amylase) is located in the culture supernatant. In contrast, the larger M23-3A.305_{—}673 α-amylase (2^{nd} α-amylase) is found from the cell surfaces.

### C. Engineering of a Deinococcus Strain with a 3-Genes Reconstructed Biomass Degradation Unit

A DNA fragment containing the α-L-Arabinofuranosidase 61_{—}237 encoding sequence (arabinofur) from strain DRH46 and a pgroESL-cat chloramphenicol resistance cassette was inserted into the chromosome of strain DG_{—}04 (amy::pTufB-amyl-amy2-pTufA-bleo), directly downstream the amy2 gene, removing pTufA-bleo and placing the arabinofur gene in the same operon as the amylases. Insertion was carried out using 500 bp fragments homologous to regions directly upstream and downstream of the pTufA-bleo cassette, therefore replacing the entire pTufA-bleo cassette. The resulting strain DG_{—}16 (amy::pTufB-amyl-amy2-arabinofur-pgroESL-cat) was checked for marker replacement (bleo to cat) and its operon structure confirmed by PCR.

### D. Engineering of a Deinococcus Strain with a 4-Genes Reconstructed Biomass Degradation Unit

Insertion of the Endocellulase Encoding Gene DRH46.66_{—}2727 (Endocell), from Strain DRH46, Downstream the Arabinofur Gene

A DNA fragment harboring the following construct (pTufA-endocell-bleo) where the Endocellulase encoding gene 66_{—}2727 from strain DRH046 is in operon with the bleo resistance gene and placed under the control of a promoter pTufA, is inserted downstream the arabinofur gene in the chromosome of strain DG_{—}16, giving strain DG-16B (amy::pTufB-amyl-amy2-arabinofur-pTufA-endocell-bleo).

### E. Engineering of a Deinococcus Strain with a 5-Genes Reconstructed Biomass Degradation Unit

An insertion cassette containing the Exocellulase encoding gene 284_{—}1-4 (exocell) from strain M1-3H followed by a sequence encoding the transcriptional unit pgroESL-sacB_{Bs}-cat [sacB, levansucrase gene from Bacillus subtilis, in operon with the cat resistance gene and under the control of the pgroESL promoter] is used for insertion in operon with the endocell gene into the chromosome of DG_{—}16B strain, resulting in strain DG_{—}16C (amy::pTufB-amyl-amy2-arabinofur-pTufA-endocell-exocell-pgroESL-sacB-cat).

### F. Engineering of Deinococcus Bacteria with an Alcohol Production Transcriptional Unit and a Biomass Degradation Unit

An ethanol production unit comprising a PDC and an ADH gene was inserted in the cells, in addition to the biomass degradation unit.

### F1.

In a first set of experiments, a cassette containing an operon with the pyruvate decarboxylase encoding gene (pdc_{Zm}) from Zymomonas mobilis and the alcohol dehydrogenase encoding gene (adh_{Zm}) from Z. mobilis, placed under the control of the pgroESL promoter and, a pTufA-bleo bleocin resistance cassette, were inserted into the chromosome of strain DG_{—}16 in place of either the endogenic pta gene (26_{—}1230) encoding Phosphate acetyl-transferase or of the entire acetate production operon containing Phosphate acetyl-transferase gene (pta, 26_{—}1230) and acetate kinase gene (ack, 26_{—}1224). The resulting strains are called DG_{—}17 (amy::pTufB-amyl-amy2-arabinofur; pta::pgroESL-pdc_{Zm}-adh_{Zm}-pTufA-bleo) and DG_{—}19 respectively.

For pta gene deletion, 500 bp fragments homologous to regions directly upstream and downstream pta gene were used as homology regions. To avoid polar effect on the neighbouring ack gene (in operon with pta gene), the first ATG codon from pta was conserved.

For insertion in place of the entire acetate production operon, 500 bp fragments homologous to regions directly upstream and downstream the ack-pta operon were used as homology regions.

The capacity and performance of these bacteria to hydrolyze starch was determined as disclosed in the experimental section. The results are summarized in Table 1 below:

| Strain | Starch hydrolysis (%) |
|---|---|
| DG | 10 |
| DG_04 | 80 |
| DG_16 | 80 |
| DG_17 | 80 |

These results show the expression of the biomass degradation unit does confer improved capacity to hydrolyze starch to the bacterium. Also, the bacterium is still viable and the reconstructed unit does not affect growth properties.

As shown table 2 below, while neither DG nor DG_{—}16 produced ethanol in the tested conditions, DG_{—}17 produces substantial amount of ethanol, not only in tubes but also in 1 L reactors.

TABLE 2

| | EtOH | EtOH | EtOH |
|---|---|---|---|
| | production | production | production |
| | (%) on wheat | (%) on wheat | (%) on wheat |
| | 3% | 6% | 6% |
| DG | 0 | 0 | — |
| DG_16 | 0 | 0 | — |
| DG_17 | 0.1 | 0.08 | 0.04 |

| | |
|---|---|
| Experiments performed in tubes | Experiment |
| | performed in 1L- |
| | bioreactors |

### F2.

In a further step of experiments, another ethanol production unit was constructed and inserted in the cells. In these experiments, following similar strategies as in F1, two new strains were engineered from DG-16, using Deinococcus DRH05 codon optimized sequences of pdc (SEQ ID NO: 16) and adh genes (SEQ ID NO: 18), giving strains DG_{—}18 (amy::pTufB-amyl-amy2-arabinofur; pta::pgroESL-pdc_{Zm}*-adh_{Zm}*-pTufA-bleo) and DG_{—}20 (amy::pTuJB-amyl-amy2-arabinofur; pta/ack::pgroESL-pdc_{Zm}*-adh_{Zm}*-pTufA-bleo).

### F3. Insertion of an NADP-Dependent ADH Encoding Gene

A metabolic flux ratio analysis showed that, in the parent DG strain, up to 56% of glucose is consumed through the Pentose Phosphate Pathway, indicating that an important part of NADPH is produced as redox potential. In order to use this NADPH pool for EtOH production, an NADP-dependent ADH encoding gene from Moorella sp HUC 22-1 is introduced into the chromosome of strains DG_{—}17 and DG_{—}18, downstream the NAD-dependent ADH gene from Z. mobilis.

These new strains, named DG_{—}21 (amy::TufB-amyl-amy2-arabinofur; pta::pgroESL-pdc_{Zm}-adh_{Zm}-adh_{Mo}-pTufA-kan) and DG_{—}22 (amy::pTufB-amyl-amy2-arabinofur; pta/ack::pgroESL-pdc_{Zm}*-adh_{Zm}*-adh_{Mo}-pTufA-kan), have been obtained by selecting the strains on kanamycine containing plates.

These strains express two different types of ADH, one dependent from NAD and one dependent from NADP. As shown table 3 below, the co-expression does maximize ethanol production. Indeed, in comparison to DG-17, strain DG-21 produced 5 times more ethanol in 1 L bioreactors as a result of expression of both ADH genes.

TABLE 3

| | EtOH | EtOH | EtOH |
|---|---|---|---|
| | production | production | production |
| | (%) on wheat | (%) on wheat | (%) on wheat |
| | 3% | 6% | 6% |
| DG | 0 | 0 | — |
| DG_16 | 0 | 0 | — |
| DG_17 | 0.1 | 0.08 | 0.04 |
| DG_21 | — | — | 0.2 |

| | |
|---|---|
| Experiments performed in tubes | Experiment |
| | performed in 1L- |
| | bioreactors |

### F4.

Completion of the biomass degradation unit by insertion of the endocellulase encoding gene DRH46.66_{—}2727 (endocell), from strain DRH46, downstream the arabinofur gene

A DNA fragment harboring the following construct (pTufA-endocell-bleo) where the Endocellulase encoding gene DRH46.66_{—}2727 is in operon with the bleo resistance gene and placed under the control of a pTufA promoter, was inserted downstream the arabinofur gene in the chromosome of strain DG_{—}21 (amy::pTufB-amyl-amy2-arabinofur; pta::pgroESL-pdc_{Zm}-adh_{Zm}-adh_{Mo}-pTufB-kan), giving strain DG_{—}23 (amy::pTufB-amyl-amy2-arabinofur-pTufA-endocell-bleo; pta::pgroESL-pdc_{Zm}-adh_{Zm}-adh_{Mo}-pTufA-kan). Insertion was carried out using 500 bp fragments homologous to regions directly upstream and downstream of the cat gene, therefore replacing the entire cat gene. Strain was checked for marker replacement (cat to bleo) and confirmed by PCR.

The endocellulase encoding gene was also inserted into strain DG_{—}22, giving strain DG_{—}24 (amy::pTufB-amyl-amy2-arabinofur-pTufA-endocell-bleo; pta::pgorESL-pdc_{Zm}*-adh_{Zm}*-adh_{Mo}-pTufA-kan).

F5. Insertion of the Exocellulase Encoding Gene M1-3H.284_{—}1-4 (Exocell), from Strain M1-3H, in Operon with the Endocell Gene

An insertion cassette containing the Exocellulase encoding gene 284_{—}1-4 (exocell) followed by a sequence encoding the transcriptional unit pgroESL-sacB_{Bs}-cat [sacB, levansucrase gene from Bacillus subtilis, in operon with the cat resistance gene and under the control of the pgroESL promoter] was used for insertion into the chromosome of DG_{—}23 and DG_{—}24, resulting in strains DG_{—}25 (amy::pTufB-amyl-amy2-arabinofur-pTufA-endocell-exocell-pgroESL-sacB-cat, pta::pgroESL-pdc_{Zm}-adh_{Zm}-adh_{Mo}-pTufB-kan) and DG_{—}26 (amy::pTufB-amyl-amy2-arabinofur-pTufA-endocell-exocell-pgroESL-sacB-cat, pta::pgroESL-pdc_{Zm}*-adh_{Zm}*-adh_{Mo}-pTufA-kan). Insertion was carried out using 500 bp fragments homologous to regions directly upstream and downstream bleo gene, therefore replacing the entire bleo gene. The resulting strain was checked by PCR for its operon structure, and for marker replacement (bleo to cat).

### G. Insertion of a Non-GMO Ethanol Producing Pathway in DG_{—}16

An ethanol production operon with the acetaldehyde dehydrogenase encoding gene (DSM22328.88_{—}978) from Deinococcus misasensis (SEQ ID NO: 11) and the alcohol dehydrogenase encoding gene (DRH46.26_{—}648) from Deinoccocus cellulosilyticus DRH46 (SEQ ID NO: 13), placed under the control of the pgroESL promoter, and the pTufA-bleo bleocin resistance cassette was inserted into the chromosome of strain DG_{—}16 in place of the endogenic pta gene (26_{—}1230) encoding Phosphate acetyl-transferase. The resulting strain is called DG_{—}29 (amy::pTufB-amyl-amy2-arabinofur; pta::pgroESL-acdh_{DSM22328}-adh_{drh46}-pTufA-bleo).

### H. Cleaning Final Modified Strains from Antibiotic Resistance Genes

To delete the pgroESL-sacB_{Bs}-cat transcriptional unit, a DNA fragment harboring the sequences of the regions flanking pgroESL-sacB_{Bs}-cat is inserted into the chromosome of strain DG_{—}25 and DG_{—}26. The same strategy is used to remove the kanamycin resistance gene from “ethanol” operon. The sacB gene from B. subtilis is used as a counter-selectable marker as it encodes for toxicity to sucrose. The final operon structures were confirmed by PCR and sequencing.

### I. Optimization of Ethanol Production by Deletion of Competing Metabolism Pathways

Deletions of 4 target genes encoding competitive activities are made in series using the following deletion/cleaning cassette. The cassette backbone contains the sacB_{Bs}-cat transcriptional unit (levansucrase gene from B. subtilis in operon with the chloramphenicol cat resistance gene) under the control of the pgroESL promoter. This cassette is inserted into the chromosome of DG strains in place the target genes (one at a time), using bleocin selection. For each insertion, a secondary DNA fragment, where the 2 chromosomic regions flanking the target locus are assembled, is used to obtain a clean deletion, removing the pgroESL-sacB-cat unit from the chromosome and counter-selecting on sucrose-containing medium.

This method is employed for the following genetic deletions:
The Alanine dehydrogenase encoding gene 31_{—}22
The Glucose dehydrogenase 1 (GDH1) encoding gene 52_{—}885
The Glucose dehydrogenase 2 (GDH2) encoding gene 22_{—}1025
The Phosphoenolpyruvate carboxykinase (PEPCK) encoding gene 52_{—}1088
The Phosphoenolpyruvate carboxylase (PEPC) encoding gene 12_{—}1095
The Malate dehydrogenase encoding gene 34_{—}424

### J. Optimized Production in Fermentors

Strain DG_{—}21 was cultivated in Infors HT multifors 2-fold 1 L fermenter system.

Cultivation conditions for both vessels were 45° C. temperature, 400 rpm stirrer speed, airflow 65 mL/min, pH at 7 was maintained by 1 M NaOH and 1.5 M H₃PO₄.

Inoculum was cultivated in shake flasks on complex medium with 10 g/L glucose. Bleocin antibiotic was added with final concentration of 6 μg/mL. Conditions for inoculum cultivations were 45° C. and 150 rpm. First cells were inoculated from glycerol stock to 250 mL Erlenmeyer flasks each containing 20 mL of the medium. After 16 h, a 1 L Erlenmeyer flasks containing 200 mL of fresh medium were inoculated at OD₆₀₀ₙₘ 0.05 and were incubated for 22 h. Adequate amounts of cell suspension from 250 mL Erlenmeyer flasks were centrifuged and re-suspended into an inoculum bottle containing 10 mL of complex medium with 10 g/L glucose. Cells were transferred from inoculum bottle to vessels with syringe.

Cultivation medium was prepared by weighting the components into a bottle and filling the bottle to the 1 L mark with cold tap water. The medium was then poured to a vessel and 1 mL of 1:10 diluted silica antifoam was added into the mix to prevent foaming during sterilization. Vessels were then autoclaved at 121° C. for 25 minutes. Cultivation media used in each vessel are described below:

| | | NH₄Cl | K₂HPO₄ | CaCl₂, 2H₂O |
|---|---|---|---|---|
| Vessel | Substrate | (g/L) | (g/L) | (g/L) |
| 1 | wheat 3% | 1 | 1 | 0.5 |
| 2 | Starch milk 20% | 1 | 1 | 0.5 |

Samplings were done manually each day through sampling line by using a sterile bottle. From these samples, several analyses were done:
Biomass quantification was done by performing qPCR method.
Free glucose was quantified using an YSI analyzer.
Gas Chromatography FID analysis (Varian CP-WAX 57 CB 25 m*0.32 mm column) was used to quantify alcohols.
Organic acids were quantified by Liquid Chromatography Mass Spectroscopy (MicrOTOF-QII Broker).

The results are provided below. They show that, in fermentors, DG_{—}21 can produce ethanol titers close to 2 g/L. Also, they show the kinetics of production is very positive, in that low glucose consumption and high cell density may be reached, further documenting the remarkable performances of the bacteria of this invention.

More particularly, FIG. 5 shows that a biomass cell density of about 7 g/L can be reached in stationary phase upon culture on wheat, and that glucose consumption continued even in stationary phase.

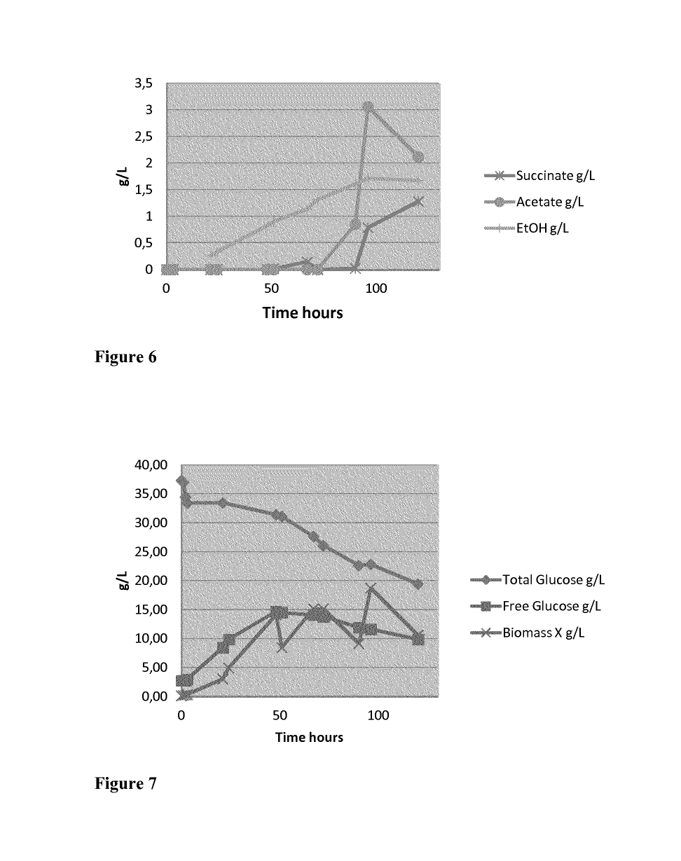

FIG. 6 shows that ethanol production was linear and reached approximately 2 g/L in 96 hours of culture. When glucose was depleted from the medium, ethanol production did not increase any further and a plateau was observed. Correlatively, succinate and acetate were detected in the supernatant, reaching respectively about 3.0 g/L and 1.2 g/L.

FIG. 7 shows that, upon culture on starch milk, a biomass cell density of about 15 g/L can be reached in stationary phase, and that glucose consumption continued even in stationary phase. After 144 h, the remaining total glucose was about 20 g/L, which indicates a low consumption rate of glucose.

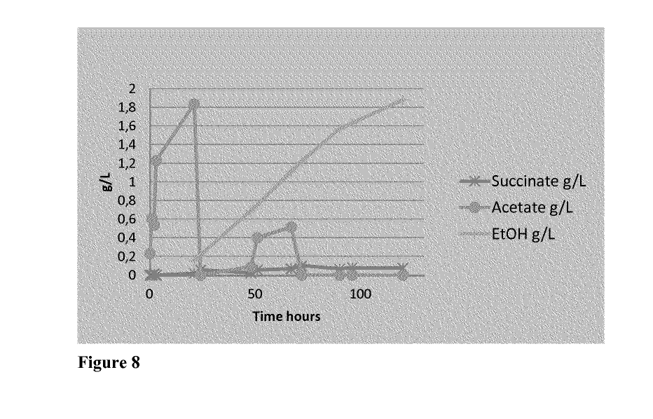

FIG. 8 shows that ethanol production increased gradually, and reached approximately 2 g/L in 144 hours culture in starch milk. Correlatively, acetate production drastically decreased.

These results therefore illustrate the performance of the bacteria to produce high levels ethanol from biomass.

1. A Deinococcus or related bacterium comprising a reconstructed biomass degradation transcriptional unit inserted in its genome, said unit comprising at least 2 genes under the control of a single promoter, said at least 2 genes encoding distinct biomass degradation enzymes involved in the degradation or hydrolysis of biomass into fermentable sugars.

2-26. (canceled)

27. The bacterium of claim 1, wherein the biomass degradation enzymes are selected from amylases, arabinofuranosidases, cellulases, xylanases, laccases, alpha-glucuronidases, and esterases.

28. The bacterium of claim 1, wherein said unit comprises three genes under the control of a single promoter, each of said three genes encoding a distinct biomass degradation enzyme selected from amylases, arabinofuranosidases and cellulases.

29. The bacterium of claim 1, wherein said unit comprises, under the control of said single promoter, 2 distinct amylase genes, or 1 amylase gene and 1 arabinofuranosidase gene, or 2 distinct amylase genes and 1 arabinofuranosidase gene.

30. The bacterium of claim 27, wherein the amylase(s) is (are) alpha amylases.

31. The bacterium of claim 27, wherein the cellulase(s) is (are) endocellulase(s) or exocellulase(s).

32. The bacterium of claim 28, wherein the arabinofuranosidase comprises SEQ ID NO: 6.

33. The bacterium of claim 1, wherein said unit further comprises at least one additional gene under the control of a second promoter.

34. The bacterium of claim 33, wherein said unit comprises (i) under the control of a single promoter, 2 amylase genes, or 1 amylase gene and 1 arabinofuranosidase gene, or 2 amylase genes and 1 arabinofuranosidase gene; and (ii) under the control of a second promoter, at least one cellulase gene.

35. The bacterium of claim 1, wherein said bacterium is a Deinococcus bacterium and said biomass degradation enzymes originate from Deinococcus bacteria.

36. The bacterium of claim 33, wherein the single or second promoter is selected from a Deinococcus pTufA promoter, pTufB promoter, or pGroESL promoter.

37. The bacterium of claim 1, wherein the reconstructed biomass degradation transcriptional unit does not contain non-Deinococcus derived nucleic acid.

38. The bacterium of claim 1, wherein the reconstructed biomass degradation transcriptional unit is inserted in the genome of said bacterium in replacement of an endogenous gene.

39. The bacterium of claim 1, wherein said bacterium further comprises a recombinant alcohol production transcriptional unit or a transcriptional unit for ethanol production.

40. The bacterium of claim 39, wherein the recombinant alcohol production transcriptional unit comprises at least one nucleic acid sequence encoding an Alcohol dehydrogenase (ADH) and/or a pyruvate decarboxylase.

41. The bacterium of claim 39, wherein the alcohol production transcriptional unit comprises at least one nucleic acid sequence encoding an NADP-dependent ADH.

42. The bacterium of claim 39, wherein the alcohol production transcriptional unit is inserted in the genome of said bacterium, optionally in replacement of an endogenous phosphate acetyl transferase gene.

43. A Deinococcus or related bacterium comprising an inactivated phosphate acetyl transferase gene.

44. A Deinococcus or related bacterium comprising at least one recombinant nucleic acid sequence encoding an NADP-dependent ADH.

45. A bacterium according to claim 1, said bacterium being selected from D. geothermalis, D. cellulolysiticus, D. radiodurans, D. proteolyticus, D. radiopugnans, D. radiophilus, D. grandis, D. indicus, D. frigens, D. saxicola, D. maricopensis, D. marmoris, D. deserti, D. murrayi, D. aerius, D. aerolatus, D. aerophilus, D. aetherius, D. alpinitundrae, D. altitudinis, D. apachensis, D. aquaticus, D. aquatilis, D. aquiradiocola, D. aquivivus, D. caeni, D. claudionis, D. ficus, D. gobiensis, D. hohokamensis, D. hopiensis, D. misasensis, D. navajonensis, D. papagonensis, D. peraridilitoris, D. pimensis, D. piscis, D. radiomollis, D. roseus, D. sonorensis, D. wulumuqiensis, D. xibeiensis, D. xinjiangensis, D. yavapaiensis or D. yunweiensis.

46. A composition comprising a bacterium according to claim 1 and at least one other bacterium.

47. An enzymatic extract of a Deinococcus or related bacterium according to claim 1.

48. A process for transforming biomass, comprising exposing a biomass to a Deinococcus or related bacterium according to claim 1, a composition comprising said Deinococcus or related bacterium or an extract thereof.

49. A process for producing an alcohol comprising exposing a sugar or biomass to a Deinococcus or related bacterium according to claim 1, a composition comprising said Deinococcus or related bacterium or an extract thereof and producing said alcohol.
